SEQUENCE LISTING (1 sequence)

SEQ ID NO: 1 - DNA, 18 bp, Bacteriophage T7
taatacgactcactatag

(19) United States
(12) Patent Application Publication
(10) Pub. No.: US 2022/0064634 A1
(43) Pub. Date: Mar. 3, 2022

(54) METHODS AND SYSTEMS FOR PROTEIN ENGINEERING AND PRODUCTION

(71) Applicant: LabGenius Ltd, London (GB)

(72) Inventors: Harrison Frederick Rickerby, London (GB); James Edward John Field, London (GB); Ekaterina Victorovna Putintseva, London (GB); Christopher Cozens, London (GB)

(73) Assignee: LabGenius Ltd, London (GB)

(21) Appl. No.: 17/610,127

(22) PCT Filed: May 11, 2020
(86) PCT No.: PCT/GB2020/051143

§ 371 Date: Nov. 9, 2021

(30) Foreign Application Priority Data
May 9, 2019 (GB) 1906566.3

(51) Int. Cl. C12N 15/10; G16B 35/10; G16B 35/20; G16B 40/20

(52) U.S. Cl. CPC C12N 15/1089; G16B 40/20; G16B 35/20; G16B 35/10

(57) ABSTRACT

The present invention provides methods for producing a protein having one or more desired properties, the method comprising: (a) a library design step, (b) a library testing step; and (c) a learning step, in which the sequence variants are each assigned a fitness score based at least in part on the result of the library testing step, and a machine learning algorithm uses the fitness score of each of the sequence variants to train a model to predict the fitness score for new sequence variants, and wherein the machine learning model trained in step (c) is used to design a new library of sequence variants. The present invention also provides a system for producing a protein having one or more desired properties, said system adapted to implement the method of the invention.

### FIELD OF THE INVENTION

Methods and systems for protein engineering and production are considered in this invention, in particular, iterative approaches for protein engineering using a combination of high-content nucleic acid libraries, high-throughput assays and artificial intelligence.

### BACKGROUND OF THE INVENTION

When engineering proteins for a specific function, one of the main challenges lies in the combinatorial explosion of possible molecules presented to the user that constitute a searchable sequence space, even when using a candidate protein as a starting point for modifications. This problem is compounded with a lack of options that can use high throughput approaches to protein engineering throughout the design-build-test-learn methodology loop that is common to synthetic biology processes. It will be appreciated that any bottle neck in the loop introduces a restriction to the exploration of the sequence space. Therefore, there exists a need to provide methods and systems that can automatically and efficiently explore the vast space of sequence variability to identify candidate proteins with a specific set of desirable properties. These and other uses, features and advantages of the invention should be apparent to those skilled in the art from the teachings provided herein.

### SUMMARY OF THE INVENTION

In accordance with the present invention, a first aspect provides a method for producing a protein having one or more desired functionalities, the method comprising:
(a) a library design step, in which a nucleic acid library comprising at least 10⁴ sequence variants is designed, wherein each sequence variant comprises a coding sequence fora protein and each sequence variant comprises at least one constant region and at least one variable region, wherein one or more constant regions are common to all sequence variants within the library, and the one or more variable regions are not common to all sequence variants within the library;
(b) a library testing step, in which the sequence variants are tested in parallel, for the one or more desired properties; and
(c) a learning step, in which the sequence variants are each assigned a fitness score based at least in part on the result of the library testing step, and a machine learning algorithm uses the fitness score of each of the sequence variants to train a model to predict the fitness score for new sequence variants;
wherein the machine learning model trained in step (c) is used to design a new library of sequence variants with an improved distribution of fitness scores.

Therefore, the methods of the invention combine a specific approach to library design, high-throughput assays and artificial intelligence to enable engineering and production of candidate proteins having one or more desired properties by efficiently exploring large areas of the sequence space.

In particular, the use of constant and variable parts enables to constrain the regions of sequence where variability would usefully be introduced, to optionally design and produce those parts separately, then to assemble these with common constant parts that contain elements like promoters and flags that are to be included in all variants. The constant parts can then easily be swapped between selected few parts having e.g. chosen flags or promoters, and combined with a library of variable parts. The variable parts can be used to efficiently explore the sequence space. Further, the use of machine learning to learn from the data obtained on the library enables to inform a new design step and therefore produce new candidate variants that can improve on the initial set of variants tested.

In embodiments, the method further comprises (a′) a library assembly step, comprising: (1) providing a first plurality of nucleic acid molecules corresponding to a first variable part of the sequence variants in the library, comprising one or more variable regions, and wherein the first plurality of nucleic acid molecules comprises variants of the one or more variable regions; (2) providing: (i) at least one further pluralities of nucleic acid molecules corresponding to at least one further variable part of the sequence variants in the library, comprising at least one further variable region wherein the at least one further plurality of nucleic acid molecules comprises variants of the at least one further variable regions; and/or (ii) at least one further plurality of nucleic acid molecules corresponding to a at least one constant part of the sequence variants in the library, each constant part comprising a constant region and no variable region, wherein the at least one further plurality of nucleic acid molecules are substantially identical; and (3) assembling each of the plurality of first and at least one further nucleic acid molecules to form the nucleic acid library, each variant in the library comprising a first variable part and at least one further part.

In embodiments, each of the plurality of nucleic acid molecules further comprises an end sequence that is identical to an end sequence of another one of the plurality other nucleic acid molecules, in order to enable the creation of overhangs for assembly of the nucleic acid molecules. In embodiments, the end sequences have a length of between 2 and 20 bases. In embodiments, the end sequences have a length of between 4 and 10 bases.

In embodiments, each sequence variant comprises at least one constant part and at least one variable part.

In embodiments, each sequence variant comprises two constant parts: a first or start part comprising a promoter sequence (e.g. a T7 promoter sequence), one or more optional tags, and the start of the coding sequence (i.e. the N-terminal part) of the encoded protein; and a second or final part containing the end of the coding sequence (i.e. the C-terminal part) of the encoded protein, and one or more optional purification tags.

In embodiments, each sequence variant comprises two variable parts, each comprising a portion of the coding sequence of the encoded protein.

In embodiments, a further constant part may be provided between the two variable parts.

In embodiments, each sequence variant has two variable parts and two constant parts. Limiting to two variable parts controls the costs associated with sourcing of variable parts, and may be useful when the variable parts comprise similar sections (e.g. repetitive scaffolds) to reduce the risk of introducing errors in the library assembly step.

In embodiments, the nucleic acid molecules corresponding to a constant part are provided as double stranded DNA. This advantageously means that the sequence can be easily manipulated and replicated, for example by PCR or by including it in a plasmid which is replicated in bacteria.

In embodiments, providing a plurality of nucleic acid molecules corresponding to a constant part comprises amplifying a nucleic acid molecule corresponding to the constant part by polymerase chain reaction.

In embodiments, the nucleic acid molecules corresponding to each of the one or more variable parts are provided as single stranded DNA, optionally wherein providing a plurality of nucleic acid molecules corresponding to the variants of one or more variable parts comprises synthesising a second DNA strand by single primer extension to form double stranded DNA. This may be particularly advantageous when complex collections of variable parts with high random variability are used, since these are difficult to synthesise with high accuracy as dsDNA.

In embodiments, providing a plurality of nucleic acid molecules corresponding to the variants of one or more variable parts comprises synthesising a second DNA strand by single primer extension to form double stranded DNA.

Advantageously, not using PCR ensures that no errors and amplification bias are introduced in the library. This is particularly advantageous when the variable parts are designed with specific probabilities of each variant, as PCR could alter these probabilities.

In embodiments, assembling each of the first plurality of nucleic acid molecules with a nucleic acid molecule from each of the further pluralities of nucleic acid molecules comprises assembling the nucleic acid molecules by USER (Uracil-Specific Excision Reagent) assembly. Without wishing to be bound by theory, it is believed that USER assembly is particularly advantageous as it is scarless, does not rely on specific recognition sequences like restriction enzymes, and results in programmable overhangs.

In embodiments, constant parts are up to about 2000 nucleotide long, and/or wherein variable parts are up to about 200 nucleotide long.

Advantageously, constant parts only have to be sourced once and can be sourced as dsDNA which can easily be replicated for example by including them in a plasmid that is replicated in bacterial cells. In embodiments, variable parts are up to about 200 nucleotides long. This may enable the variable sequences to be synthesised chemically with high accuracy, including where highly complex collections of variable sequences are to be generated.

In embodiments, each sequence variant comprises a plurality of constant parts and/or a plurality of variable parts.

In embodiments, the library design step (a) comprises defining fully the sequence of each of the one or more constant parts.

In embodiments, the library design step (a) comprises designing at least one of the one or more variable regions to include random variability in at least one position, optionally wherein the library design step (a) comprises designing at least one of the one or more variable regions to include random variability in one or more specific positions of the at least one variable region.

In embodiments, the random variability is constrained by providing a probability for each base (A, C, T, G). In embodiments, the random variability is constrained by providing a probability for each amino acid. In embodiment, the probabilities for each base may be the same across each of the variable positions, or may be dependent on the variable position. In embodiments, the probability for at least one base at least one position may be 0.

In embodiments, the library design step (a) comprises designing at least one of the one or more variable parts to include random variability in one or more specific positions of the variable part(s).

In particular, including random variability may comprise constraining the variability to sequences that correspond to a DNA codon.

In embodiments, including random variability comprises constraining the variability to sequences that do not correspond to a stop codon. This may enable exclusion of sequence that may encode truncated protein, thereby focusing the exploration of the sequence space to areas more likely to be of practical use.

In embodiments, the library design step (a) comprises: selecting a nucleic acid sequence encoding for a protein that has at least one of the one or more desired properties; automatically identifying one or more regions of the sequence where variability is expected to result in an improvement of the at least one of the one or more desired properties and/or acquisition of at least one of the one or more desired properties; and defining the one or more variable parts to include the one or more regions of the sequence where variability is expected to result in an improvement of the at least one of the one or more desired properties and/or acquisition of at least one of the one or more desired properties.

In some embodiments, the library design step (a) further comprises: identifying one or more regions of the sequence where variability is expected to be detrimental to the integrity of the protein and/or to at least one of the one or more desired properties; and defining one or more of the one or more constant regions to include the one or more regions of the sequence where variability is expected to be detrimental to the integrity of the protein and/or to at least one of the one or more desired properties.

In embodiments, at least one of the one or more constant regions comprises one or more sequences selected from: a promoter sequence, an enhancer sequence, a localisation signal, a flag sequence, a marker sequence, a ribosome binding site, a stop codon, a start codon, a 5′ stem loop structure, a 3′ stem loop culture, an origin of replication and a selection sequence.

In embodiments, the method further comprises a step (a) of producing the proteins encoded by each sequence variant to obtain a protein library, wherein the library testing step (b) comprises subjecting the protein library to one or more assays to test for the one or more desired properties. The nucleic acid library may be a DNA library and producing the protein library may comprise transcribing and translating the DNA library. In embodiments, transcribing the DNA library comprises incubating the DNA library with a T7 RNA polymerase. The use of the T7 RNA polymerase may be advantageous as this polymerase has a well-defined promoter sequence and a very low error rate.

In embodiments, the method further comprises a step (a″) of producing the proteins encoded by each sequence variant to obtain a protein library, wherein the library testing step (b) comprises subjecting the protein library to one or more assays to test for the one or more desired properties. The nucleic acid library may be a DNA library and producing the protein library may comprise transcribing and translating the DNA library. In embodiments, transcribing the DNA library comprises incubating the DNA library with a T7 RNA polymerase. The use of the T7 RNA polymerase may be advantageous as this polymerase has a well-defined promoter sequence and a very low error rate.

In embodiments, the nucleic acid library is a DNA library and producing the protein library comprises transcribing and translating the DNA library, wherein translating the library comprises synthesising RNA-polypeptide fusion molecules each comprising an RNA sequence variant bound to the protein that it encodes. In embodiments, this is done using a technique called “mRNA display”. In embodiments, this is done using a technique called “phage display”. Without wishing to be bound by theory, it is believed that mRNA display is advantageous in the context of the invention because the entire process occurs in vitro. This removes the need to transform the DNA library into cells, which is often a low efficiency process, thereby creating a bottleneck and potentially biasing the library. Further, in mRNA display, the coding sequence is covalently linked to the protein, thereby preventing the two parts from dissociating even under harsh testing conditions. This may enable a wide range of desired properties to be tested, including e.g. resistance to harsh conditions.

In embodiments, the nucleic acid library is a DNA library and producing the protein library comprises transcribing and translating the DNA library, wherein translating the library comprises propagating phage that display a coat protein-polypeptide fusion wherein the polypeptide fused to the coat protein corresponds to a sequence variant of the DNA library. In embodiments, this is done using a technique called “phage display”. Without wishing to be bound by theory, it is believed that phage display is advantageous in the context of the invention because it allows for more efficient display of larger proteins (for example, proteins larger than 10 kDa, for example, 10-100, 10-50, 15, 30, 40 or 50 kDa) compared to mRNA display, thus allowing for more efficient selection of variants within a library.

In embodiments, the protein library produced is quality controlled by extracting the proteins and performing a reverse transcription quantitative PCR to quantify the amount of mRNA associated with the protein library.

In embodiments, the protein library is produced from the nucleic acid library entirely in vitro.

In embodiments, the library testing step (b) comprises separating the protein library into at least 2 samples depending on the results of the one or more assays, and sequencing the nucleic acids present in at least one of the at least 2 samples.

In embodiments, each sample is subject to a reverse transcription step, and a purification step to extract the DNA part of the sample, before DNA sequencing.

This approach may enable the use of next-generation sequencing to identify functionally distinct groups of proteins. As a result, the method is able to identify the proteins that do/do not have a desired functionality (depending on how they perform in the assays) at a very high throughput. Identification of the variants at the protein level would be extremely error prone (e.g. mass spectrometry proteomics is currently still significantly noisier than DNA sequencing) and/or significantly slower.

In embodiments, the method further comprises barcoding the nucleic acids present in at least 2 of the at least 2 samples and sequencing the at least 2 barcoded samples together.

In embodiments, the learning step (c) comprises aligning the sequences obtained by sequencing with the sequences designed in step (a), and quantifying the number of times that each sequence appears in each sample.

In embodiments, at least one of the constant regions comprises a sequence that encodes for a protein purification tag, optionally wherein the protein purification tag is a streptavidin binding peptide. Advantageously, this may enable streptavidin coated beads to be used for separation of the proteins after translation, to perform quality control of the mRNA display step, or to perform some assays such as protease stability assays.

In embodiments, the one or more desired properties is/are chosen from: physico-chemical properties of the proteins, activity-related properties, physiologically-relevant properties, and pharmacokinetic properties.

In embodiments, physico-chemical properties may be chosen from chemical stability (e.g. resistance to oxidants, acids, etc.), solubility, thermal resistance, resistance to drying and rehydration, etc.

In embodiments, activity-related properties may be chosen from enzymatic activity, specificity of any activity or binding, off target effects (i.e. activity or binding to targets other than primary target), binding affinity, association/dissociation rates for a chosen target, ability to inhibit or stimulate an enzyme, avidity (functional affinity) etc.;

In embodiments, physiologically-relevant properties may be chosen from protease resistance, immunogenicity, ability to activate one or more immune effector(s), ability to cross the blood-brain barrier, ability to cross epithelia (e.g. gut epithelia, lung epithelia, etc.), ability to enter cells, ability to cross cellular membranes/lipid bilayers, ability to enter cells of a specific cell type, ability to penetrate solid tumors, suitability for organ/cell-type specific delivery.

In embodiments, pharmacokinetic properties may be chosen from elimination half-life, clearance, toxicity, organ specific pharmacokinetics, etc.

In embodiments, at least one of the constant regions comprises a sequence that encodes for a protein purification tag, optionally wherein the protein purification tag is located at the C-terminus of the protein, wherein one of the one or more desired properties is protease resistance and running the protein library through one or more assays comprises exposing the protein library to one or more proteases, purifying the proteins using the protein purification tag and identifying the sequence variants that are not cleaved by the one or more proteases.

In embodiments, the protein purification tag is located at the C-terminus of the protein.

Advantageously, when using mRNA display, the mRNA associated with each protein will be located at the N-terminus of the protein. Therefore, sequence variants that are not cleaved by the one or more proteases will still be attached to their mRNA, whereas sequence variants that are cleaved will not. As such, when the proteins are purified, the mRNAs of the cleaved variants will be washed off, and only the protease-resistant variants will be sequenced.

In embodiments, one of the one or more desired properties is binding to a specific target, and the library testing step (b) comprises incubating the protein library with the specific target immobilised on a surface and separating the protein library into a sample that is bound to the surface and a sample that is not bound to the surface.

In embodiments, the method further comprises washing the surface after incubation to remove non-specific interactions. In embodiments, the method further comprises exposing the same library to control conditions (e.g. the surface only, without the immobilising target), to filter out false-positives (e.g. variants that bind to the surface rather than the target).

In embodiments, the library testing step comprises testing the variants for a plurality of properties, and the learning step comprises assigning a plurality of fitness scores to each variant tested, wherein each fitness score corresponds to one of the plurality of properties, wherein the learning step comprises training a plurality of machine learning algorithms, wherein each machine learning algorithm is trained to predict at least one of the plurality of fitness scores for new sequence variants.

In embodiments, the learning step comprises assigning a combined fitness score for each sequence variant tested, wherein the combined fitness score for each sequence variant tested is based on the plurality of fitness scores for the sequence variant.

In embodiments, the one or more fitness scores associated with each sequence variant depends on the number of times that each sequence appears in a first sample and the number of times that each sequence appears in a second sample, optionally wherein the first sample corresponds to a sample that is deemed to have a positive result in one of the one or more assays, and the second sample is a control sample.

Advantageously, this method of scoring the sequences may enable to reduce the impact of noise in the system. If a sequence only appears once after selection, this could simply be an error introduced during library preparation, or a sequence that happened to not encounter a protease, rather than it actually having increased stability.

In embodiments, a fitness score associated with a sequence variant is a score that quantifies how biased a particular step is in regard to a sequence. For example, an assay to test for a desired functionality can be associated with a score (also referred to as “bias” or “bias score”) which quantified how biased the step is towards each of the sequences in the library by comparing sequencing data (e.g. sequence counts) on the library before and after the assay.

In embodiments, the score is quantified between 0 (strong negative bias) and 1 (strong positive bias) using a Bayesian methodology. Intermediate scores may be regarded as negatively biased, positively biased or “similar to before” (which might be labelled “successful” in some contexts) depending on a subjective confidence level.

In embodiments, the Bayesian methodology used is designed to quantify, for a given sequence, the expectation to measure y counts after the step, assuming a Poisson distribution with an unknown mean λ, and after measuring x counts before the step (i.e. p(y|x)).

In embodiments, p(y|x) may be calculated as (N2/N1)^{y}*((x+y)!/(x!y!(1+(N2/N1))^{(x+y+1)})), where x is observed from a sample size N1 and y is observed from a sample size N2.

Advantageously, this approach reflects the assumption that we can have a higher confidence in the bias of a step in relation to a sequence variant when the sequence is observed many times after the step, compared to a situation where the variant is observed only a few times.

In embodiments, the score may be used to define a group of sequences that is “negatively biased” (for example with bias score <0.1), a group of sequences that is “positively biased” (for example with bias score >0.9), with the remaining sequences being defined as “as expected/not biased”. These definitions may be used to train the machine learning algorithm in the learning step.

In embodiments, the thresholds for sequences being negatively biased or positively biased may be set using a chosen confidence level CL. In particular, sequences with a score >1−ε may be labelled as “positively biased”, whereas sequences with a score <c may be labelled as “negatively biased”, where ε is calculated as (1−CL)/2. In embodiments, CL is at least 0.9975, at least 0.955 or at least 0.683.

In embodiments, a fitness score is only calculated for a sequence variant if the sequence appears at least one in each of the first and second samples. This may be useful to exclude sequences that appear due to mistakes in the sequencing process and are not “true reads”.

In embodiments, the scores are filtered to exclude sequence variants that appear less than a chosen number of times in the first sample, the second sample, or the sum of the first and second samples. For example, a threshold of minimum 10 reads across both samples may be applied.

In embodiments, a separate bias score may be calculated for each sequence variant, for each desired functionality. For example, assuming that the protein library is subjected to a first assay to quantify binding affinity to a first target, and a second assay to quantify binding affinity to a second target, two separate scores may be calculated, reflecting the bias of each of these assays in relation to each sequence variant.

In embodiments, the first sample corresponds to a sample that is deemed to have a positive result in one of the one or more assays, and the second sample is a control sample. Suitably, a control sample is a sample that is deemed to have a negative result in one of the one or more assays, or a sample corresponding to the library prior to the one or more assays used to qualify the first sample as having a positive result.

In embodiments, the machine learning algorithm is a classifier, wherein the machine learning algorithm is a neural network.

In embodiments, the machine learning algorithm is a regression algorithm. For example, the algorithm may utilise lasso (Least Absolute Shrinkage and Selection Operator) regression, ridge regression (also referred to as Tikhonov regularization), or logistic regression. In other words, the machine learning algorithm may be trained to build a model that can predict a numerical value (e.g. a continuous numerical value) for each sequence. Without wishing to be bound by theory, it is believed that classifiers may be particularly appropriate when the data indicates that the bias scores strongly cluster around the ends of the range of scores (i.e. the majority of the sequence variants have a bias score close to 0 or close to 1).

In embodiments, the machine learning algorithm is a neural network. In specific embodiments, the machine learning algorithm is a convolutional neural network.

In embodiments, the machine learning algorithm is a multiple classifier system. That is to say, the algorithm is an ensemble of classifiers. For example, an ensemble algorithm.

In embodiments, the machine learning algorithm is a support-vector machine algorithm.

Advantageously, the classifier is able to predict a score for any new sequence that is fed into the model. As such, it can be used to optimise a population of sequences using various optimisation methods. Therefore, an optimisation process is performed to identify a new population of sequences that has an improved fitness (for example, an improved distribution of fitness scores) compared to the sequences that have been tested thus far (for example, compared to the sequence variants within a “parent” library or population).

A library or population of sequence variants having an “improved distribution of fitness scores” may be one wherein the distribution of the one or more fitness scores of the sequence variants is skewed towards a more positive value compared to the distribution of one or more fitness scores of the sequence variants within a parent library or population of sequences. That is to say, the optimization process provides a new library or population of sequence variants that have average fitness score(s) (for example, 1, 2, 3, 4, 5, 6, 7 or more fitness scores corresponding to 1, 2, 3, 4, 5, 6, 7 or more desired properties) that is higher than the average fitness score of a parent library or population of sequences variants that has not undergone an optimisation process (for example, the parent library or population of sequence variants that directly precedes the new, optimised, library or population of sequence variants).

In one embodiment, a library or population of sequence variants having an “improved distribution of fitness scores” is one wherein the one or more mean fitness scores of the sequence variants is higher than the one or more mean fitness scores of the sequence variants within a parent library or population of sequence variants. Additionally, or alternatively, a library or population of sequence variants having an improved fitness may be one wherein one or more median fitness scores of the sequence variants is higher than the one or more median fitness scores of the sequence variants within a parent library or population of sequence variants parent library or population of sequence variants. Additionally, or alternatively, a library or population of sequence variants having an improved fitness may be one wherein one or more modal fitness scores of the sequence variants is higher than the one or more modal fitness scores of the sequence variants within a parent library or population.

In another embodiment, a library or population of sequence variants having an “improved distribution of fitness scores” is one that contains a smaller proportion of non-functional sequence variants compared to a parent library or population. For example, less than 50% (for example, less than 50, 40, 30, 20, 15, 10, 7, 5, 2, or less than 1%) of variants in the library or population of sequence variants are non-functional sequence variants (for example, said non-functional sequence variants do not display one or more improved desired properties, for example, improved physicochemical proprieties, improved activity related properties and/or improved physiologically relevant properties). Preferably, less than 20% (for example, 20, 19, 18, 17, 16, 15, 14, 13, 12, 11, 10, 9, 8, 7, 6, 5, 4, 3, 2 or less 1%) of variants in the library or population are non-functional sequence variants. More preferably, less than 10% of variants in the library or population are non-functional sequence variants.

In another embodiment, a library or population of sequence variants having an “improved distribution of fitness scores” is one that contains a higher proportion of variants that display one or more improved fitness scores (for example, a higher proportion of variants display one or more improved desired properties, for example, improved physicochemical proprieties, improved activity related properties and/or improved physiologically relevant properties) compared to a parent library or population of sequence variants. For example, the top at least 1% (for example, at least 1, 2, 5, 7, 10, or at least 20%) of sequence variants have one or more improved desirable property compared to the top at least 1% (for example, at least 1, 2, 5, 7, 10 or at least 20%) of variants in a parent library or population.

In another embodiment, a library or population of sequence variants having an “improved distribution of fitness scores” is one wherein the variant with the highest fitness score(s) in said library or population has [a] higher fitness score(s) compared to the variant with the highest fitness score(s) within a library or parent population. That is to say that, the variant with the highest fitness score(s) in the optimised library or population is one that displays one or more improved fitness scores (for example, one or more improved desired property, for example, improved physicochemical proprieties, improved activity related properties and/or improved physiologically relevant properties) compared to the variant with the highest fitness score(s) in a parent library or population.

Additionally, or alternatively, a library or population of sequence variants having an “improved distribution of fitness scores” is one containing at least one variant wherein one or more variable regions have a sequence similarity (DNA and/or amino acid sequence) of less than 99% (for example, less than 98, 97, 96, 95, 90, 85, 80, 75, 70, 60, 50, 40, 30, 20, 10 or less than 5%) with respect to the corresponding one or more variable regions of all, or a proportion of, the variants within a parent library or population. Additionally, or alternatively, the library or population of sequence variants having an “improved distribution of fitness scores” may be one containing at least 5%, for example, at least 10, 15, 20, 25, 30, 35, 40, 45, 55, 65, 70, 75, 85, 90, 95 or 100% of variants that have one or more variable regions having a sequence similarity (DNA and/or amino acid sequence) of less than 99% (for example, less than 98, 97, 96, 95, 90, 85, 80, 75, 70, 60, 50, 40, 30, 20, 10 or less than 5%) with respect to the corresponding one or more variable regions of all, or a proportion of, the variants within a parent library or population.

In embodiments, a library or population of sequence variants having an “improved distribution of fitness scores” is one containing at least one variant wherein one or more variable regions have a sequence similarity (DNA and/or amino acid sequence) of less than 99% (for example, less than 98, 97, 96, 95, 90, 85, 80, 75, 70, 60, 50, 40, 30, 20, 10 or less than 5%) with respect to the corresponding one or more variable regions of all, or a proportion of, the variants within a parent library or population, and display one or more improved fitness scores (for example, at least one variant displays one or more improved desired properties, for example, improved physicochemical proprieties, improved activity related properties and/or improved physiologically relevant properties) compared to the variant contained in a parent library or population with the highest fitness score(s).

In embodiments, a library or population of sequence variants having an “improved distribution of fitness scores” is one containing at least one variant wherein one or more variable regions have a sequence similarity (DNA and/or amino acid sequence) of less than 99% (for example, less than 98, 97, 96, 95, 90, 85, 80, 75, 70, 60, 50, 40, 30, 20, 10 or less than 5%) with respect to the corresponding one or more variable regions of all, or a proportion of, the variants within a parent library or population, and wherein said variants of the library or population having an improved distribution of fitness scores display one or more improved fitness scores (for example, said variants display one or more improved desired properties, for example, improved physicochemical proprieties, improved activity related properties and/or improved physiologically relevant properties) compared to one or more fitness scores displayed by all, or a proportion, of variants of a parent library or population.

In embodiments that refer to “all, or a proportion of, the sequence variants” of a library or population, it is to be understood that “all the sequence variants” of a library or population refers to substantially all the variants of a library or population. Further, it is to be understood that “a proportion of the sequence variants” of a library or population refers to less than substantially all the variants of a library or population, for example, 95, 90, 85, 80, 75, 70, 60, 50, 40, 30, 20, 10, 5, 2, 1% or less than 1%) of the variants of a library or population.

For the avoidance of doubt, the term “parent library or population” refers to a library or population of sequence variants that has undergone less optimisation compared to a new population of sequences.

That is to say, the parent library or population may be one that directly precedes the new, optimised, library or population. For example, the parent library or population may have undergone at least n−1 (for example, n−1, n−2, n−3 or n−4, wherein n is the number of optimisation rounds that the new library has undergone) optimization rounds compared to the new library or population. Preferably, the parent library or population is one that has undergone n−1 optimization round compared to the new library or population (i.e. the parent library or population is one that directly precedes the new, optimised, library or population). More preferably, a parent library or population is prepared according to the library design step (a) of the present invention.

In embodiments, the machine learning model trained in step (c) is used to design a new library of sequence variants by iteratively optimising a library of sequence variants in silico, optionally wherein the library of sequence variants is iteratively optimised using a genetic algorithm.

In embodiments where the machine learning algorithm is a classifier, the machine learning algorithm can be used to build a model that predicts the class of any new sequence that it is provided, and/or that provides continuous values representing the probability for a new sequence that it is provided to belong to any of the defined classes. In embodiments where the machine learning algorithm is a regression algorithm, the machine learning algorithm can be used to build a model that can predict a score for any new sequence that it is provided.

In embodiments, the machine learning algorithm can be used to predict a class, score or probability of belonging to a class for an initial population of sequence variants, and this information can be used to obtain a new population to be provided to the machine learning algorithm.

In embodiments, the learning phase comprises calculating a distance between a new library and any previously generated library (e.g. any previously tested library and/or any previous in silico library). In embodiments, the distance between sequence libraries is calculated using the Jensen-Shannon divergence method.

In embodiments where a plurality of fitness scores are calculated for each sequence variant, a multiobjective optimisation may be performed, which aims to optimise a library of sequence variants for each of the fitness scores jointly.

In embodiments, the library of sequence variants is iteratively optimised using a genetic algorithm.

In embodiments, the parameters of the genetic algorithm are optimized to favor exploration of the search space at the beginning of the optimization. Parameters of the genetic algorithm that are optimised may include one or more of: a choice of crossover strategy, crossover rate, mutation strategy, mutation rate, number of parents, population size, number of elites in the population, selection methods, etc.

In embodiments, the library of sequence variants may be optimised using Markov Chain Monte Carlo (MCMC) methods and/or optimization algorithms, such as gradient descent. Such algorithms and methods are known in the art.

In embodiments, the new library of sequence variants is derived from a subset of the variants tested in step (b).

In embodiments, a subset of the library (referred to as the initial population, or generation 0) is run through the classifier, and each sequence is assigned a fitness score. The subset is then mutated using a genetic algorithm, to obtain a first generation, which is fed back into the classifier. This process is repeated until a library with a sufficiently high fitness is generated, or a maximum, number of iterations is reached. These parameters can be predefined by a user or can be assigned default values

In embodiments, the method further comprises repeating steps (a) to (c) with the new library.

In embodiments, the method comprises repeating steps (a) to (c) up to 10 times in total with new libraries.

In embodiments, the method comprises repeating steps (a) to (c) until a predetermined criteria is met, such as a specific value of one or more desired properties, for at least 1, preferably at least 3, at least 5 or at least 10 variants in the library.

In embodiments, step (c) comprises training the machine learning algorithm using the one or more fitness scores of any previously tested sequence variants.

In embodiments, the new library is derived from a subset of the variants tested in the immediately preceding step (b) or any preceding step (b).

In embodiments, the new library includes variants that were not present in previous libraries. For example, the new library may include variants predicted to have high fitness scores. In embodiments, the new library does not include variants previously tested.

In embodiments, the new library comprises at least one sequence variant encoding for a protein with the one or more desired properties.

According to a second aspect, there is provided a system for producing a protein having one or more desired properties, the system comprising: (i) a processor adapted to implement any of the methods described herein including any of the methods according to embodiments of the first aspect; (ii) a laboratory automation apparatus, wherein the apparatus is controlled by the processor so as to implement at least the testing step.

In embodiments, the laboratory automation apparatus comprises one or more of the group consisting of: liquid handling and dispensing apparatus; container handling apparatus; a laboratory robot; an incubator; plate handling apparatus; a spectrophotometer; chromatography apparatus; a mass spectrometer; thermal-cycling apparatus; nucleic acid sequencing apparatus; and centrifuge apparatus.

According to a further aspect, the invention relates to library of sequence variants obtained using the methods described herein.

In embodiments, the library of sequence variants is a nucleic acid library. In embodiments, the library is a DNA library. In embodiments, the library of sequence variants is a peptide or protein library (for example, a peptide ligand library, an antibody library, an antibody mimetic library, or antibody fragment library, for example a single chain antibody or single domain (i.e. a VHH domain).

In embodiments, a sequence variant has one or more variable regions, for example at least one, two, three or four variable regions (for example, 3, 4, 5, 6, 7, 8, 9, 10, 11, 12, 13, 14, 15, 20, 25, 30, 35, 40, 45 or 50 variable regions).

In embodiments, each variable region may independently be 1 to 200 or, 1 to 100, 1 to 60 nucleotides long, for example, 1 to 3, 3 to 6, 6 to 9, 9 to 12, 12 to 15, 15 to 18, 18 to 21, 21 to 24, 24 to 27, 27 to 30, 30 to 33, 33 to 36, 36 to 39, 39 to 42, 42 to 45, 45 to 48, 48 to 51, 51 to 54, 54 to 57 or 57 to 60 nucleotides long. Preferably, 1 to 100, 1 to 60, 1 to 48, 3 to 45 or 3 to 30 nucleotides long. A variable region could be a single nucleotide.

In embodiments, the one or more variable regions may independently be 1 to 60 or 1 to 20 amino acids long, for example, 1, 2, 3, 4, 5, 6, 7, 8, 9, 10, 11, 12, 13, 14, 15, 16, 17, 18, 19 or 20 amino acids long. Preferably, 1 to 15 or 1 to 10 amino acids long. A variable region could be a single amino acid.

According to a further aspect, there is provided a container comprising the library according to the previous aspect.
According to yet another aspect, there is provided a protein having one or more desired properties, wherein the protein is obtained using the methods described herein.

In embodiment, the protein comprises one or more constant parts and one or more variable parts. In embodiments, the one or more constant parts comprise scaffold domains. In embodiments, the one or more variable parts comprise interaction mediating domains.

### BRIEF DESCRIPTION OF THE DRAWINGS

FIG. 1 is a flowchart of iterative protein engineering strategy according to one embodiment of the invention;

FIG. 2 shows an example of a library structure according to embodiments of the invention;

FIG. 3 shows an example of a protease stability assay according to embodiments of the invention;

FIG. 4 shows an example of a binding assay according to embodiments of the invention;

FIG. 5 illustrates a calculated bias score according to embodiments of the invention, for three different values of the number of reads observed for a particular variant before an assay is performed to separate out variants of a library that have a desired functionality (x=2, x=20, x=200), as a function of the ratio of: the number of reads observed for the particular variant in the subset of the library after the assay (y) to the number of reads observed for the variant before the assay (x);

FIGS. 6A-6E show the results of an example of a library selection process according to embodiments of the invention, wherein a library of variants is expressed using phage display and selected for resistance to protease and binding to a target using 3 consecutive rounds of selection, the population of variants being sequenced after each round; in particular, FIG. 6A shows the total number of raw reads in each sequencing run (prior to selection, labelled as ‘pre’ and after each round of selection, labelled as ‘round_1’, ‘round_2’ and ‘round_3’), FIG. 6B shows the total number of variants present in the population before selection (‘pre’) and after each round of selection, FIG. 6C shows the number of variants present in the population before selection (‘pre’) and after each round of selection, relative to the total number of reads (see FIG. 6A) for the corresponding sequencing run, FIG. 6D shows the total number of variants present in the population before selection (‘pre’) and after each round of selection, excluding any variants that were not present in the starting library, and FIG. 6E shows frequency tables showing the change in library composition at various variable positions before (‘pre’) and after each of 3 rounds of selection (‘round_1’, ‘round_2’ and ‘round_3’)—excluding those mutations that were not present in the original library;

FIGS. 7A and 7B show the results of an example of a library selection process according to embodiments of the invention, wherein a library of variants is expressed using mRNA display and selected for resistance to proteases (trypsin (FIG. 7A) and chymotrypsin (FIG. 7B)), the population of variants being quantified by qPCR after selection; in particular, FIGS. 7A and 7B show the results of the qPCR quantification (ct value, number of cycles at which the fluorescence signal reaches a level above background) for flow through samples (FT) and samples captured on the beads (Beads) for each of the three libraries;

FIGS. 8A to 8C shows the results of an example of a library optimisation process according to embodiments of the invention; in particular, FIGS. 8A to 8C show for specific iterations (FIG. 8A shows the starting population, FIG. 8B shows the population at iteration 6, and FIG. 8C shows the population at iteration 14), the fitness score distribution of the current population (continuous curve) and of the initial population (histogram) on the left panel, the distribution of variants in the library of the current iteration (middle panel), and the pareto-front (maximum average fitness score for two separate parameters) for a number of libraries (right panel);

FIG. 9 shows the Spearman correlation between actual and predicted fitness of a population of sequences is R=0.67, which demonstrates that the model is able to accurately predict binding to the target of interest based only on amino acid sequence; and

FIG. 10 shows the activity of candidate molecules in a cell-based potency assay. The candidate molecules tested were predicted to be high performing variants using machine learning as described herein. 68% of the candidate molecules that the model predicted to have improved potency compared to the original molecule displayed improved potency in the cell-based potency assay.

### DETAILED DESCRIPTION OF THE INVENTION

All references cited herein are incorporated by reference in their entirety. Unless otherwise defined, all technical and scientific terms used herein have the same meaning as commonly understood by one of ordinary skill in the art to which this invention belongs.

Unless otherwise indicated, the practice of the present invention employs conventional techniques of chemistry, molecular biology, microbiology, recombinant DNA technology, and chemical methods, which are within the capabilities of a person of ordinary skill in the art. Such techniques are also explained in the literature, for example, M. R. Green, J. Sambrook, 2012, Molecular Cloning: A Laboratory Manual, Fourth Edition, Books 1-3, Cold Spring Harbor Laboratory Press, Cold Spring Harbor, N.Y.; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press; Durbin R., Eddy S., Krogh A. and Mitchinson G. (1998), Biological sequence analysis, Cambridge University Press; David W. Mount (2004), Bioinformatics, Cold Spring Harbor Laboratory Press. Each of these general texts is herein incorporated by reference.

Prior to setting forth the invention, a number of definitions are provided that will assist in the understanding of the invention.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

As used herein, the term “library” or “library of sequence variants” refers to a collection of related nucleic acid or polypeptides (also referred to herein as “peptides” or “proteins”) that differ from each other in at least one position of their sequence. A nucleic acid library therefore comprises a collection of nucleic acids, typically DNA molecules, that differ from each other in at least one base. In the context of the invention, each nucleic acid sequence variant comprises the coding sequence for a protein. Therefore, a protein library according to the invention contains a collection of proteins that have been obtained by expressing a nucleic acid library. As the skilled person would understand, such a protein library may contain molecules that differ from each other in at least one amino acid residue, as well as molecules that do not differ from each other, due to redundancy in the genetic code. Further, as the skilled person would understand, a sample comprising a library may in fact contain a plurality of copies of some or all of the sequence variants.

In embodiments, a nucleic acid library comprises at least 10⁴ sequence variants, preferably at least 10⁵ or at least 10⁶ sequence variants. In embodiments, a nucleic acid library comprises at least 10⁷, at least 10⁸, at least 10⁹, or at least 10¹⁰ sequence variants. As will be further described below, sequence variants may be obtained by introducing random variability in a chosen starting sequence or set of related sequences. A set of related sequences may for example comprise a single sequence defined with flexibility at certain positions (e.g. position p may be x or y), or a set of sequences corresponding to e.g. homologues and/or orthologues. As such, a library of 10⁶ sequence variants does not necessarily comprise 10⁶ different sequences. Instead, a library of 10⁶ sequence variants may comprise 10⁶ sequences that each result from a sampling of the pool of sequences that is possible within the constraints defined for introducing variability in the starting sequence(s). In practice, the number of different sequences in the library may be upwardly limited by the constraints imposed on the variability introduced in the starting sequence(s) as well as the length of the starting sequence(s). In embodiments, the total number of different sequences in a nucleic acid library may be at least about 10k, at least about 50k, at least about 100k, or at least about 150k.

In the context of the invention, as will be described further below, sequence variants in a nucleic acid library comprise one or more constant regions and one or more variable regions, wherein one or more constant regions are common to all variants in the library, and the one or more variable regions are not common to all variants in the library. Sequence variants may be provided as a plurality of parts that are assembled to form each sequence variant in the library. When using a plurality of parts, each part may be a constant part (if it does not contain variable regions), or a variable part (if it contains at least one variable region). When designing a nucleic acid library, constant parts/regions, also referred to herein as “fixed parts/regions” are defined completely. As such, the sequence of nucleotides that make up a constant part/region may be fully defined and common to all sequences in the library. Alternatively, it is also possible for multiple equivalent constant parts/regions to be present in a library, but each such constant part/region is defined fully at inception when the library is designed, and is not randomly varied.

In the context of the invention, the term “high-throughput” relates to assays, processes and protocols that are capable of processing in parallel all of the variants of a nucleic acid library or corresponding protein library as described above.

As used herein, a “fitness score” (also referred to as “score” or “bias” or “bias score”) is a score that is associated with a sequence variant in a protein or nucleic acid library, and that represents the likelihood of the variant having one or more desired properties.

The invention provides a novel methodology that uses the combination of large nucleic acid library design, high-throughput assays and machine learning to engineer proteins with a desired functionality.

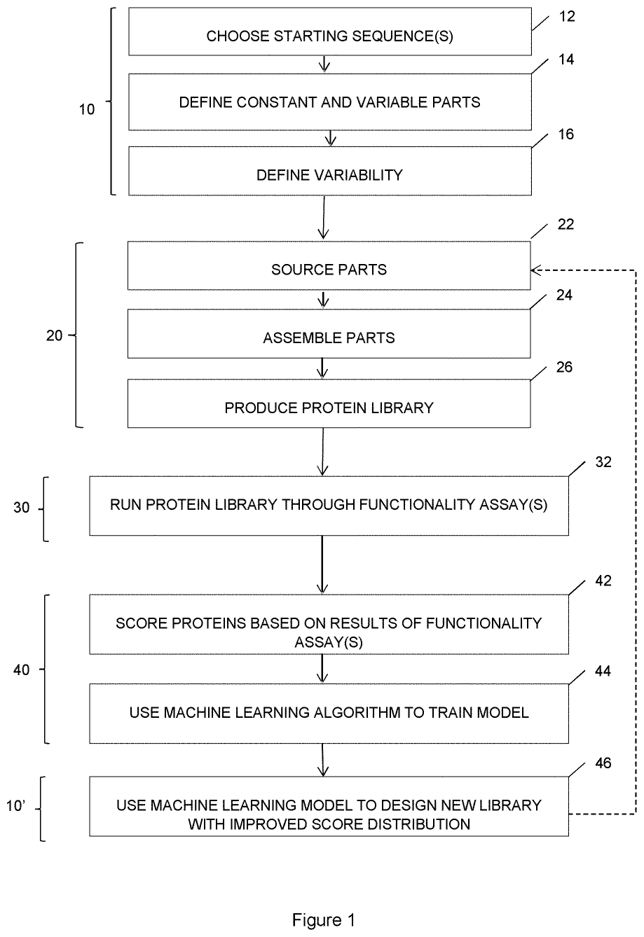

FIG. 1 shows a flow chart of a method for producing a protein having one or more desired properties according to an embodiment of the present invention. At a high level, the illustrated method comprises a library design step 10, a library build step 20, a library testing step 30, and a learning step 40, where the result of the learning step 40 is used to inform a new library design step 10′, which can then optionally be used as an input to a new cycle of build 20, test 30 and learn 40. In the illustrated embodiment, the library design step 10 includes designing a nucleic acid library of sequence variants by choosing 12 a starting sequence or set of sequences, defining 14 constant and variable regions in the starting sequence (or across the set of starting sequences), and defining 16 the variability to be introduced in the variable region(s). For example, a starting sequence may be chosen because it already has at least one of the one or more desired properties, or has the potential to be adapted to have at least one of the one or more desired properties. In the illustrated embodiment, the library build step 20 includes sourcing 22 physical parts that will be used to build the library, assembling 24 the parts to obtain the nucleic acid library, and producing 26 a protein library from the nucleic acid library. Parts that do not contain variable regions are referred to herein as “constant parts”. Parts that contain at least one variable region are referred to herein as “variable parts”. A sequence variant in the nucleic acid library may be formed by assembly of multiple parts, at least one of which is a variable part. A sequence variant will typically contain at least one variable part. Additional variable parts and constant parts may advantageously be provided, depending on the relative size and location of the variable and constant regions. For example, where large constant regions are present, these may advantageously be provided as separate constant parts. By contrast, relatively small constant regions interspersed between variable regions may be advantageously provided as parts of variable parts. In the library testing step 30, all the sequence variants in the protein library are tested 32 in parallel, for one or more properties. In the learning step 40, the sequence variants tested in step 30 are assigned 42 one or more fitness scores based at least in part on the result of the library testing step 30. The fitness scores of the sequence variants are used to train 44 one or more models using a machine learning algorithm to predict the one or more fitness scores for new sequence variants. The machine learning model(s) trained in step 44 is/are then used to design 16 a new library of sequence variants with an improved fitness scores distribution. In embodiments, the design 10, 10′ and learn 40 steps are performed in silico, while the build 20 and test 30 steps involve physical parts and are typically performed in vitro. However, depending on the nature of the assays that are performed in step 32, some of the test step 30 may be performed in silico. For example, sequence variants may be analysed using one or more in silico assays to e.g. predict the likelihood of the sequence variants having one or more desired properties.

Desired properties may be chosen from physico-chemical properties of the proteins, such as chemical stability (e.g. resistance to oxidants, acids, etc.), solubility thermal resistance, resistance to drying and rehydration (e.g. retain acceptable level of activity or other function following drying and rehydration), etc; activity-related (e.g. “functional”) properties such as enzymatic activity, specificity of any activity or binding, off target effects (i.e. activity or binding to targets other than primary target), binding affinity, association/dissociation rates for a chosen target (kₒₙ, k_{off}, k_{D}), ability to inhibit or stimulate an enzyme, avidity (functional affinity) etc.; physiologically-relevant properties, such as protease resistance, immunogenicity, ability to activate one or more immune effector(s), ability to cross the blood-brain barrier, ability to cross epithelia (e.g. gut epithelia, lung epithelia, etc.), ability to enter cells, ability to cross cellular membranes/lipid bilayers, ability to enter cells of a specific cell type, ability to penetrate solid tumors, suitability for organ/cell-type specific delivery, etc.; pharmacokinetic properties such as elimination half-life, clearance, toxicity, organ specific pharmacokinetics, etc. Properties that may be assessed in silico may include protein stability, immunogenicity, binding affinity, or any other functionality that is at least partially derivable from in silico sequence analysis. Each of these steps will now be examined in more detail.

Designing the nucleic acid library as described above, by specifying constant and variable regions enables to constrain the exploration of the protein sequence space to specific areas (i.e. those that are represented by the variable parts). This in turn simplifies the protein engineering process and allows it to be focused for example in areas where variability is likely to result in an improvement in relation to the one or more desired properties. Further, when the variants in the library are structurally defined in terms of parts, some of which can be constant parts and some of which can be variable parts, these can be sourced separately, then assembled. This may result in a significant practical and cost efficiency improvement since constant parts only have to be sourced once for the library and can then be amplified as desired (such as via PCR), and the sourcing of a plurality of variable parts can be limited to specific (preferably short) regions of sequences. Further, constant parts can be designed to include functional elements such as promoters, flags, enhancers, localisation signals, markers, parts of the protein sequence that serve as e.g. scaffold, etc., which are common to all of the sequences in the library. Additionally, alternative versions of the constant parts can be simply obtained (for example including different promoter or flag) and combined with a collection of variable parts to create a new library.

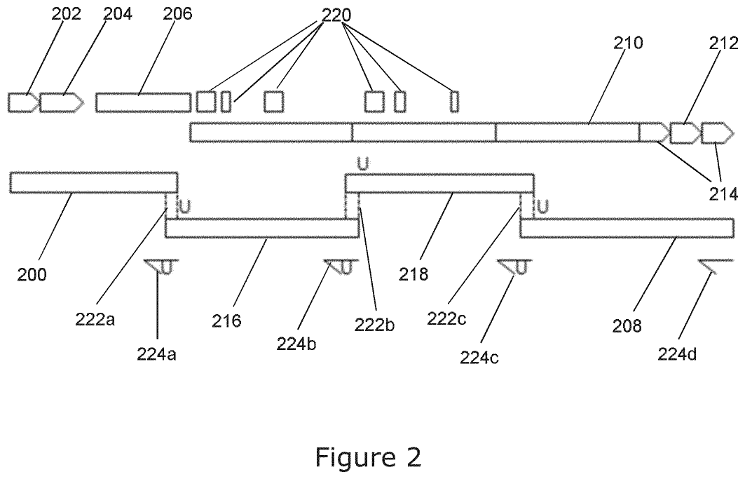

FIG. 2 shows an example of a library structure according to embodiments of the invention, and illustrates the results of the steps 12, 14 and 16 above. In the embodiment shown on FIG. 2, each sequence variant comprises a first constant part 200 comprising a promoter 202 and a tag 204 (for example, a purification tag), where the whole of the constant part represents a constant region of the sequence. The first constant part 200 includes a portion of the N-terminal cap 206 of the encoded protein. Each sequence variant further comprises a second constant part 208, comprising a portion of the C-terminal cap 210 of the encoded protein, and a purification tag 212 surrounded by linker sequences 214. Each sequence variant further comprises two variable parts 216, 218. Each variable part 216, 218 includes a at least one variable region 220 each comprising a subset of a plurality of positions where variability is introduced. Each of the parts 200, 208, 216, 218 further comprises at least one short end sequence 222a, 222b, 222c that is identical to an end sequence of an adjacent part, to allow for the creation of overhangs for assembly.

In embodiments, the short sequence (and corresponding overhangs) may have a length of between 2 and 20 bases. In embodiments, the short sequence (and corresponding overhangs) may have a length of between 4 and 10 bases. FIG. 2 further shows primers 224a, 224b, 224c, 224d, each of which is provided to anneal with one of the parts 200, 208, 216, 218, in order to generate a double stranded DNA part from a single DNA part by PCR extension of the primer. In the illustrated embodiment, some of the primers, specifically the primers 224a, 224b, 224c that bind to regions of the parts that fall within the short end sequences 222a, 222b, 222c that are identical between pairs of adjacent parts, contain a deoxyuridine. This may be useful for the assembly step 24, as will be explained further below. Briefly, the presence of a deoxyuridine in these primers will lead, upon extension, to the creation of double stranded DNA fragments corresponding to parts 200, 216 and 218, each containing a U at one end, which can be recognized by a Uracil-Specific Excision Reagent to create a ‘sticky end’ or overhang for assembly. In the embodiment shown on FIG. 2, parts 216, 218 and 208 contain a deoxyuridine adjacent to the short end sequences 222a, 222b and 222c (respectively in part 216, 218 and 208). This may be useful for the assembly step 24, as explained above and further below. In embodiments, complementary primers may be provided to amplify the constant parts 200 and 208. In other words, although only reverse primers 224a, 224d are illustrated in FIG. 2, corresponding forward primers may be provided to allow for PCR amplification of the constant parts using a pair of primers for each constant part. Similarly, corresponding forward primers may be provided to amplify the variable parts. These may advantageously contain deoxyuridine. Without wishing to be bound by theory, it is believed that amplification of constant parts may be advantageous in order to obtain a pool of constant parts for combination with various variable parts. By contrast, amplification of variable parts may advantageously be avoided for example in order to reduce the risk of introducing biases in the library by artificially enriching it with some sequences.

In embodiments, constant parts are designed to be up to about 2000 nucleotides long. As explained above, constant parts advantageously only have to be sourced once and do not contain variability. As such, these sequences can easily be sourced as double stranded DNA (dsDNA), which can advantageously be replicated at low cost, for example by including them within a plasmid that is capable of replication in bacterial cells. In embodiments, variable parts are designed to be up to about 200 nucleotides long. Such lengths are advantageously suitable to be synthesised chemically with high accuracy. Further, variable parts can be sourced as single stranded DNA (ssDNA). This may be particularly advantageous in contexts where complex collections of variable parts with high random variability are used, since these are difficult to synthesise using traditional overlap extension PCR.

As shown in the embodiment of FIG. 2, the variable regions are often located within the coding sequence of a protein encoded by the variants in the library. As such, variable parts typically comprise a portion of the coding sequence of the protein encoded by the variants in the library. At least one constant region is typically provided, which comprises a promoter sequence (e.g. a T7 promoter sequence), a ribosome binding site, one or more optional tags, and the start of the coding sequence (i.e. the N-terminal part) of the encoded protein. Depending on the size of the constant region, this may advantageously be provided as a constant part. In embodiments, a variable region may instead or in addition contain non coding sequences that are expected to have a regulatory function. For example, a variable part may be provided which comprises some or part of a promoter sequence, ribosome binding site, etc. Such embodiments may advantageously be used to investigate whether variability in these regions can have a desired effect on expression of the coding sequence of the protein encoded by the variants in the library. Further, at least one second or final constant part may be provided containing the end of the coding sequence (i.e. the C-terminal part) of the encoded protein, and one or more optional purification tags. In embodiments, constant parts may comprise one or more sequences encoding functional elements, for example: an enhancer sequence, a localisation signal, a flag sequence, a marker sequence, and a selection sequence.

Although the embodiment shown on FIG. 2 comprises two variable parts and two constant parts, it will be appreciated that a multiplicity of other combinations of parts are possible. In particular, a further constant part may be provided between two variable parts. Alternatively, no constant part may be provided. For example, all of the parts provided may comprise one or more variable regions, which may be flanked by/adjacent to constant region(s). Further, constant regions may be advantageously divided in more than one constant part. This may be advantageous for example when very large sequences are used, and/or where modularity in the functional elements provided in the constant parts may be advantageous. In embodiments, each sequence variant has exactly two variable parts and two constant parts. Without wishing to be bound by theory, it is believed that limiting the library structure to two variable parts controls the costs associated with sourcing of variable parts, and may be useful when the variable parts comprise similar sections (e.g. repetitive scaffolds) to reduce the risk of introducing errors in the library assembly step.

In step 16, a variability to be introduced in the library is defined. In embodiments, variable regions are designed to include a random variability in at least one position. The position (or a plurality of positions) may be defined (as in the case of positions 220 shown in the embodiment of FIG. 2), or may be random throughout the variable region (as would be the case for example using random mutagenesis). Therefore, in embodiments, variable regions are designed to include a random variability in one or more specific positions of the variable region(s). The random variability (whether specific or random in its position) may be constrained by providing a probability for each base (A, C, T, G). In embodiments where multiple specific variable positions are used, the probabilities for each base may be the same across each of the variable positions, or may be dependent on the variable position. In embodiments, the probability for at least one base at least one position may be 0 (i.e. one or more specific bases may be excluded). In embodiments, variability may be constrained so as to limit the variable sequences to sequences where each triplet of the sequence corresponds to a DNA codon. In specific embodiments, variability may be constrained so as to exclude variants that include stop codons within the variable parts, so as to remove sequences that potentially encode truncated proteins. In embodiments, variability may be constrained so as to make some codons less likely to occur than others, for example by assigning weights to codons. For example, codons that encode certain amino acids, such as cysteine and proline may be preferably avoided but not formally excluded, for example by applying lower weights to codons encoding these amino acids than to other codons (which may for example be assigned default weights). In embodiments, variability may be constrained by assigning weights to codons designed to ensure that the ratio of amino acids that would appear in the protein library encoded by the variants approximately corresponds to a desired ratio.

In embodiments, the variable regions may be designed by analysing the chosen protein sequence(s) to identify one or more regions where variability is expected to result in an improvement/acquisition of at least one desired properties. In embodiments, such regions may be identified by aligning protein sequences related to the chosen protein sequence to: identify conserved regions, non-conserved regions being deemed to be variable by default, and/or identify functional regions (sometimes referred to as ‘domains’) such as interaction regions/domains which could be varied e.g. to change the interaction partner. In embodiments, such regions may be identified by structure analysis of the chosen protein (using an experimental or predicted protein structure) to identify interaction regions, exposed regions, weakness points, etc. In embodiments, such regions may be identified by sequence analysis to identify potential weakness points (e.g. protease sensitivity points such as exposed loop). In embodiments, such regions may be identified by literature analysis. In embodiments, the variable regions may be designed using models obtained by applying machine learning algorithms to data associated with one or more previously obtained library(ies). Such models may be used in order to identify one or more regions where variability is expected to result in an improvement/acquisition of at least one desired properties, and may additionally be used to identify specific mutations or combinations of mutations to be included or excluded when introducing variability in the library. As the skilled person would understand, any combinations of each of these approaches may be combined within one library design process, which can additionally be at least partially automated. Conversely, in embodiments, the constant regions may be designed by identifying one or more regions of the chosen sequence(s) where variability is expected to be detrimental to the integrity of the protein and/or to at least one of the one or more desired properties. This can be performed using any of the above approaches.

In the assembly step 24, nucleic acid molecules corresponding to each of the constant parts (if present) and nucleic acid molecules corresponding to the variants of one or more variable parts, which are separately sourced in step 22 (for example, sourced from commercial oligonucleotide synthesis services), are physically assembled to create each of the nucleic acid sequence variants of the library. Prior to assembly, a plurality of nucleic acid molecules corresponding to each of the one or more constant parts (if using) may be obtained by amplifying each of the one or more constant parts by polymerase chain reaction (PCR) as known in the art. Further, prior to assembly, a plurality of double stranded nucleic acid molecules corresponding to the variants of one or more variable parts may be obtained by synthesising a second DNA strand by single primer extension. Advantageously, by not using PCR to produce the variable parts ensures that errors and amplification bias are not introduced into the library. This is particularly advantageous when the variable parts are designed with specific probabilities for each variant, as normal variations in the fidelity and amplification bias of PCR could alter these probabilities. Assembly of the constant and variable parts into a combined double stranded nucleic acid sequence can be performed using any assembly method known in the art.

In embodiments, assembling the parts comprises assembling the parts by USER (Uracil-Specific Excision Reagent) assembly. USER assembly works by incorporating a non-natural nucleotide base called deoxyuridine (closely related to uridine) into the nucleic acid parts of the library at specific positions. Therefore, in such embodiments, the nucleic acid parts include deoxyuridine residues at specific points in their sequence. These can be introduced by PCR and/or can be present in the ssDNA part and/or the primer used for single primer extension. Deoxyuridines in the parts are then processed by the USER enzyme mix, which first chops out the base of the deoxyuridine, then cleaves the DNA backbone either side of the deoxyuridine. This allows the short ends (e.g. 3′ ends) of the molecules to dissociate (on account of their low melting temperature), leaving a short single stranded region. These single stranded regions then hybridise with the complementary strands on corresponding input parts. Finally, the DNA backbones are sealed using a DNA ligase enzyme (for example, T4 ligase).

USER assembly is advantageous because it does not rely on restriction enzymes, is scarless and results in programmable overhangs. Restriction enzymes recognise specific sequence motifs in the DNA. When using highly randomised libraries, these motifs are likely to occur within the coding sequence of the library, thereby destroying some variants. Further, many traditional methods of DNA assembly leave “scars”, which are short fixed sequences that always occur when the regions are assembled. This is problematic when the scars are present in functional sequences such as protein coding sequences. Finally, USER assembly uses regions of complementary single stranded DNA at the termini of the fragments to be assembled (called “sticky ends”), which direct assembly. This is also the case in many other methods, but with USER assembly, the sequence and the length of the sticky ends is not built into the process itself and can be designed with the single constraint that the sequence must allow incorporation of deoxyuridine residues where a strand will be cut to generate a sticky end on the complementary strand. As such, the specificity (including directionality) and efficiency of the assembly process can be designed. Therefore, in embodiments, the library design step 10 comprises designing the constant (if using) and variable parts to allow for the later incorporation of deoxyuridine residues to form sticky ends (overhangs) for the assembly step.

In embodiments, step 24 comprises using the Darwin assembly method. The Darwin assembly method is known in the art. For example, Cozens et al., 2018 (Nucleic Acids Res; 46(8): e51, which is incorporated herein by reference) describes a protocol for assembling a library using the Darwin assembly method. The present inventors have found that the use of Darwin assembly in the method of the present invention allows for efficient addition of large numbers (for example, more than 3, for example, 4, 5, 6, 7, 8, 9, 10, 11, 12, 13, 14, 15, 20, 25, 30, 35, 40, 45 or 50) of small variable regions (for example, variable regions that are 1 to 15, 1 to 30, 1 to 50, 1 to 75, 1 to 100 or 1 to 200 nucleotides long, preferably less than 100 nucleotides long) in the DNA library. Furthermore, the present inventors have found that the use of Darwin assembly in the present method reduces non-specific insertion or deletion of bases in the library variants, which reduces the incidence of frameshift mutations. The present inventors have found that Darwin assembly is particularly useful for introducing variable regions throughout a binding protein, for example antibody framework regions and antibody mimetic framework/scaffold regions.

In embodiments, step 24 comprises using inverse PCR. The inverse PCR method is known in the art, for example, see Ochman et al., 1989 (Erlich H. A. (eds) PCR Technology. Palgrave Macmillan, London). Inverse PCR is a particularly simple technique that allows for rapid and efficient assembly of simple DNA libraries, since it requires just one PCR amplification step to introduce the intended mutations from a template. The present inventors have found that inverse PCR is particularly effective in the method of the present invention when the library design is simple, (i.e. there is a small region of variability, e.g. a single nucleotide, or regions of about 3 to 50 nucleotides long, for example 3 to 30 nucleotides long and/or a small number of regions of variability, e.g. less than 10, less than 5, 4, 3 or less than 2, e.g. a single region of variability).

Before testing of the library for desired properties can be performed, a protein library is obtained from the nucleic acid library in step 26. As the nucleic acid library is typically a DNA library, this includes transcribing and translating the DNA library. In embodiments, at least one of the constant parts is designed to include a T7 promoter, and transcribing the DNA library comprising incubating the DNA library with a T7 RNA polymerase. Advantageously, the T7 RNA polymerase has a well-defined promoter sequence (TAATACGACTCACTATAG (SEQ ID NO:1), where the transcription begins at the G, which is at the 3′ end), and has a very low error rate.

According to the invention, the nucleic acid library is preferably translated in such a way as to maintain a relationship between each RNA template and its encoded protein, i.e. by using a so called “display technology”. Advantageously, this means that the protein library can be subjected to high-throughput assays that relate to protein functions in step 30 (i.e. where at least a significant part of the library is tested in parallel), while enabling high throughput identification of the proteins identified to have one or more desired properties as a result of the assays. In embodiments, translating the nucleic acid library to produce the protein library comprises synthesising RNA-polypeptide fusion molecules each comprising an RNA sequence variant bound to the protein that it encodes. In embodiments, this may be done using a technique called “mRNA display”. In a particular embodiment, a modified oligonucleotide comprising a puromycin (a small molecule antibiotic) is attached to the end of the transcribed mRNA template. This is performed by ligating a piece of DNA with a 3′ puromycin molecule (referred to as a “puromycin linker”) to the 3′ end of each mRNA template. The piece of DNA comprises a secondary structure which stalls translation, thereby allowing the puromycin to enter into the ribosome and become covalently linked to the peptide that is being synthesised. As such, upon translation, the puromycin will form a covalent bond between the protein being assembled and the mRNA. The presence of the mRNA may alter the results of the assays used to test for the desired properties, particularly if the protein is small. However, this potential disadvantage is outweighed by the benefits associated with ease of identification of the protein variants (see below).

In embodiments, other display technologies may be used, as known in the art, such as any display technology reviewed in Galan et al., Mol. BioSyst., 2016, 12, 2342-2358, the content of which is incorporated herein by reference. For example, any display technology selected from phage display, CIS display (cis-activity based display), cDNA display, yeast display, E. coli display, ribosome display, covalent antibody (CAD) display, in vitro compartmentalization, spore surface display and SNAP-tag display may be used. In one embodiment, the display technology used is selected from the group consisting of mRNA display or phage display.

Without wishing to be bound by theory, it is believed that phage display is advantageous in the context of the invention because it allows for efficient display of large proteins (for example, proteins larger than 10 kDa, for example, 15, 30, 40 or 50, 10-100 or 10-50 kDa) compared to mRNA display, thus allowing for more efficient selection of variants within a library of corresponding to [a] large protein(s). Additionally, without wishing to be bound by theory, it is believed that mRNA display is advantageous in the context of the invention because the entire process occurs in vitro. This removes the need to transform the DNA library into cells, which is often a low efficiency process, thereby creating a bottleneck and potentially biasing the library. Further, in mRNA display, the coding sequence is covalently linked to the protein, thereby preventing the two parts from dissociating even under harsh testing conditions. This enables a wide range of desired properties to be tested, including e.g. resistance to harsh conditions. In embodiments, the protein library produced may be quality controlled by purifying the proteins in the sample and performing a reverse transcription quantitative PCR to quantify the amount of mRNA associated with the protein library. In such embodiments, at least one of the constant region may be designed to comprise a sequence that encodes for a protein purification tag. For example, the protein purification tag may be a streptavidin binding peptide. If the mRNA display step was successful, this analysis should show a presence of RNA in the protein library sample after protein purification.

In embodiments, wherein phage display is used as the display technology, the phage display selection process is performed using a range of selection stringencies. For example, selection stringencies suitable for use in the present invention include, for example, varying target protein concentration, varying protease concentration (for example trypsin and/or chymotrypsin concentration), varying target protein concentration and protease concentration (for example trypsin and/or chymotrypsin concentration).

Having obtained a protein library at step 26, the protein library may now be run through one or more assays to test for the one or more desired properties. The assays may enable to separate the protein library into at least 2 samples. Because the protein library was obtained in a manner that preserves the relationship between a nucleic acid sequence and its encoded protein (for example using mRNA display), one or both these two samples can therefore be subjected to next-generation sequencing. In embodiments, for example when mRNA display is used, this includes reverse transcribing and purifying any sample to be sequenced. The use of next-generation sequencing to identify proteins in samples that have been characterised using the one or more functional assays enables identification of the proteins that do/do not have a desired functionality (depending on how they perform in the assays) at a very high throughput. Identification of the variants at the protein level would be extremely error prone (e.g. mass spectrometry proteomics is currently still significantly noisier than DNA sequencing) and/or significantly slower. In embodiments, two or more samples that have been separated can be barcoded and sequenced together. In embodiments, after sequencing, the sequences read (also referred to as “reads”) may be aligned with the sequences of the nucleic acid library designed in step 10 (or 10′, as the case may be). In embodiments, the reads may be aligned with the sequence design that was used to generate the library, rather than to a set of sequences explicitly enumerating all of the possible combinations of parts in the library. This may advantageously impact the computational efficiency of the alignment process. In this context, the “sequence design” may refer to separate sequences for each part in the library (rather than each possible combination of parts in the library), and/or to a generic sequence (or set of generic sequences) that allows for variability (optionally constrained variability) in any region that was designed as a variable region, when aligning the reads. After alignment, the reads may be merged into continuous sequences. Preferably, a sequencing technology is used that provides long reads, such as e.g. in the order of one to a few hundred base pairs, or about 600 base pairs long. Advantageously, a paired-end sequencing technology may be used. For example, a paired-end sequencing technology with reads that are one to a few hundred base pairs long (for example about 300 base pairs long) may be advantageous. For example, Illumina® bead-based sequencing technologies such as those used in the MiSeq system may be used. Advantageously, the use of long reads may increase the likelihood of being able to uniquely attribute reads to sequence variants, even when some sequence variants may share a subset of variable regions. Depending on the length of the sequence variants and/or of the parts used, sequencing technologies that provide even longer reads, for example in the order of one to 50 thousands of base pairs may be used. For example, single molecule real time sequencing technologies such as those in the Sequel System from PacBio may be used. The reads and/or merged sequence may be subject to one or more quality control steps, such as by applying filters on scores associated with the base calling process, whether on a per-position basis, or an average across multiple positions (e.g. entire reads or sliding windows). The number of times that each sequence appears in each sample may then be counted (also referred to as “count”). In embodiments, as will be further described below, the library may also be sequenced prior to the step of subjecting the library to one or more assays. This may enable a comparison of the library composition prior to and following an assay designed to select for one or more desired properties.

In embodiments, the one or more desired properties is/are chosen from: binding to a specific target, protease resistance, stability at chosen physicochemical conditions, etc.

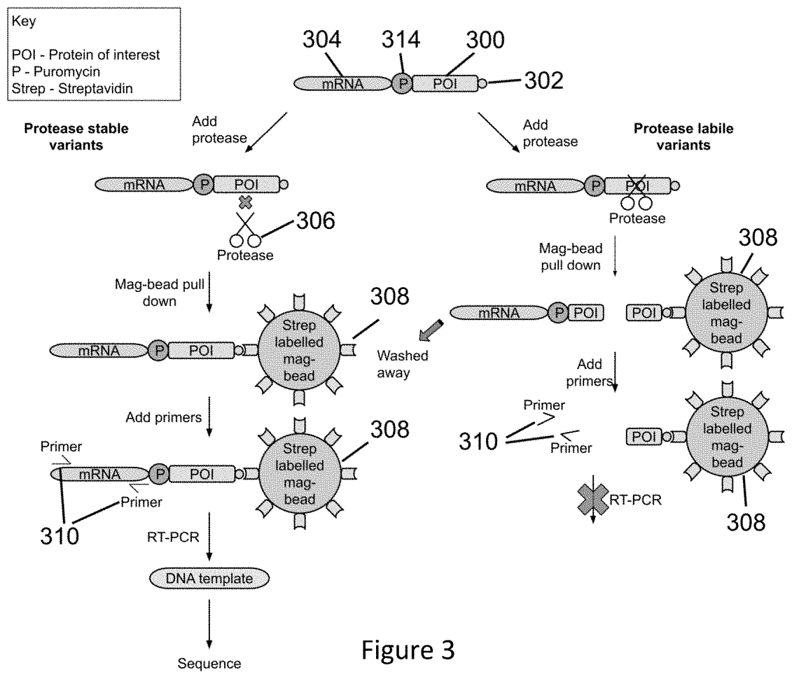

FIG. 3 shows an example of a protease stability assay according to embodiments of the invention. For protease stability assays according to embodiments of the invention, the nucleic acid library is designed such that the encoded proteins 300 (shown as “protein of interest” or POI, on FIG. 3) comprise a protein purification tag 302 at their C-terminus. For example, a protein purification tag may be a streptavidin binding peptide (for example, a “strep-tag”). Following mRNA display, the mRNA template molecule 304 associated with each protein will be bound to the N-terminus of each protein 300 in the protein library, via the puromycin molecule 314. The protein library is digested with one or more proteases 306. After a defined period of time, the proteins are purified using an appropriate affinity purification method. In the embodiment shown on FIG. 3, this is performed using magnetic beads 308 labelled with streptavidin. Since all proteins 300 are strep-tagged at the C-terminus, they bind these magnetic beads 308. The C-terminus of proteins that have been cleaved by proteases will still bind these beads, but their coding mRNA strand 304 will be washed away during the immobilisation process. This way, any template RNA 304 that is left on the bead belongs to protease stable variants. This RNA can then be then reverse transcribed using primers 310 to obtain a corresponding DNA molecule 312. The DNA molecule 312 can then be sequenced to uncover which proteins are protease stable. In embodiments, RNA that is washed away during the magnetic pull down can also be reverse transcribed and sequenced to give a negative data set to compare to the positive set.

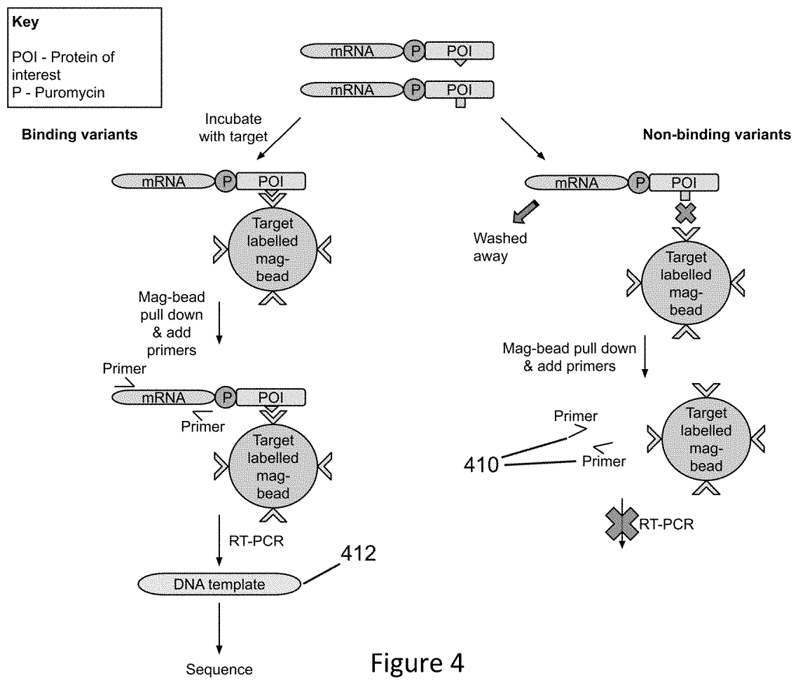

FIG. 4 shows an example of a binding assay according to embodiments of the invention. Following mRNA display, the protein library may contain encoded proteins 400 (shown as “protein of interest” or POI, on FIG. 4) that have a binding domain 402a and encoded proteins 400 (shown as “protein of interest” or POI, on FIG. 4) that have a binding domain 402b, where each protein 400 is associated with its mRNA template 404 via the puromycin molecule 414. The library can therefore be incubated with a specific target 306 immobilised on a surface, which in the embodiment shown on FIG. 4 is the surface of a magnetic bead 408. The proteins that have a binding domain 402a that binds to the target 306 can be separated (for example by pulling down the magnetic beads) from the proteins that have a binding domain 403b that does not bind the target 408. RNA in the first sample can then be reverse transcribed using primers 410 to obtain the corresponding DNA 412. These can then be sequenced to identify sequence variants that bind to the target 306. In embodiments, the method may further comprise washing the surface after incubation, in order to remove non-specific interactions. In embodiments, the method further comprises exposing the same library to control conditions (e.g. the surface only, without the immobilising target), to filter out false-positives (e.g. variants that bind to the surface rather than the target).

At step 42, one or more fitness scores may be associated with each variant tested in step 32. In particular, the library testing step may comprise testing the variants for a plurality of properties, and a plurality of fitness scores may be assigned to each variant tested, wherein each fitness score corresponds to one of the plurality of properties. The scoring process will now be described in more detail. In embodiments, the one or more fitness scores associated with each sequence variant depends on the number of times that each sequence appears in a first sample and the number of times that each sequence appears in a second sample, where this number can be obtained as explained above, by subjecting each sample to next-generation sequencing. Indeed, without wishing to be bound by theory, this is underlined by the assumption that the more frequently a sequence appears in a certain pool, the more likely it is that this sequence truly belongs to that pool. For example, if a sequence appears 100 times more frequently after being exposed to proteases during protease selection (compared to before protease selection), it will receive a high score for protease stability, where sequences that appear 100 times less frequently after being exposed to proteases during selection will receive a low score for protease stability. Advantageously, this method of scoring the sequences enables to reduce the impact of noise in the system. If a sequence only appears once after selection, this could simply be an error introduced during library preparation, or a sequence that happened to not encounter a protease, rather than it actually having increased stability.

In embodiments, a fitness score associated with a sequence variant is a score that quantifies how biased a particular step is in regard to a sequence. This may for example be a probabilistic score, as explained below. The score may be associated with any step in the method, but is more commonly associated with any sub-step (e.g. a functionality assay) of the testing step. For example, an assay to test for a desired functionality can be associated with a score (also referred to as “bias” or “bias score”) which quantifies how biased the step is towards each of the sequences in the library by comparing sequencing data (e.g. sequence counts) on the library before and after the assay.

In embodiments, the score is quantified between 0 (strong negative bias) and 1 (strong positive bias). For example, this may be performed using simple ratio based approaches (e.g. based on computing a count ratio) or Bayesian methodologies. The use of a score between 0 and 1 may be beneficial for use in many models such as e.g. regression models. In embodiments, the score is quantified between 0 (strong negative bias) and 1 (strong positive bias) using a Bayesian methodology. In embodiments, continuous scores between 0 and 1 may be used to train a model, as will be explained further below. In embodiments, continuous scores between 0 and 1 may be assigned with labels, for example for the purpose of training classifiers. For example, intermediate scores may be regarded as negatively biased, positively biased or “similar to before” (which might be labelled “successful” in some contexts) depending on a subjective confidence level. In embodiments, one or more confidence levels may be defined to label scores as “below expectation/failure” (e.g. below a first threshold), “above expectations/success” (e.g. above a second threshold) or “within expectations” (e.g. between the first and second thresholds). In embodiments, the score is quantified using a Bayesian methodology designed to quantify, for a given sequence, the expectation to measure y counts for a sequence variant after the step, assuming a Poisson distribution with an unknown mean A, and having measured x counts for the sequence variant before the step (i.e. p(y|x)). In particular, p(y|x) may be calculated as (x+y)!/(x!y!2(x+y+1)) if the sample sizes from which x and y are drawn are equal. If the sample sizes from which x and y are drawn are not even (x is observed from a sample size N1 and y is observed from a sample size N2), p(y|x) may be calculated as (N2/N1)y*((x+y)!/(x!y!(1+(N2/N1))(x+y+1))). These values assume that p(x) and p(y) come from the same Poisson distribution with an unknown mean A, where a flat prior is assumed for A. Further details on these statistics can be found in Audic & Claverie (Genome Research 1997, 7:986-995), which is incorporated herein by reference. In embodiments, a non-flat prior may be assumed for A. For example, as explained in Audic & Claverie (Genome Research 1997, 7:986-995), a limited region of interest for A may be chosen instead of 0 to infinity (i.e. flat prior).

The score of a sequence variant may then be derived, by calculating the sum of all p(yᵢ|x) where yᵢ is any count y in the subset [0,y]. This advantageously results in a score between 0 and 1.

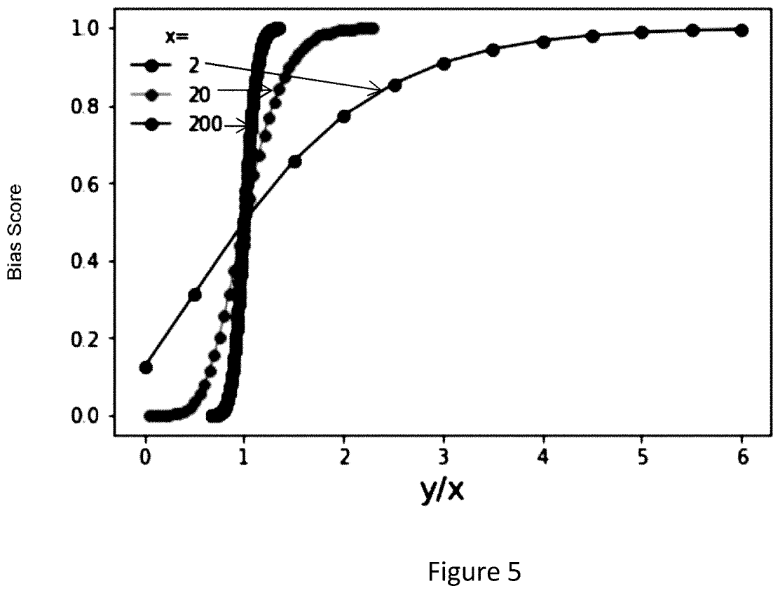

FIG. 5 illustrates the calculated bias score for N2/N1=1.02 for three different values of the number of reads observed for a particular variant before the step (x=2, x=20, x=200), as a function of the ratio of the number of reads observed for the particular variant after the step (y) to the number of reads observed for the variant before the step (x). As can be seen on FIG. 5, this scoring approach is such that the larger the value of x (i.e. the more the sequence was observed prior to the step), the quicker the bias score asymptotes to the extremes (0 for negatively biased, 1 for positively biased). Advantageously, this reflects that we a higher confidence in the bias of a step can be obtained in relation to a sequence variant when the sequence is observed 40 times after the step and 20 times before the step, compared to a situation where the variant is observed twice before the step and 4 times after the step.

In embodiments, the score may be used to define a group of sequences that is “negatively biased” (for example with bias score <0.1), a group of sequences that is “positively biased” (for example with bias score >0.9), with the remaining sequences being defined as “as expected/not biased”. These definitions may be used by the machine learning algorithm in step 44, as will be further described below. In embodiments, the thresholds for sequences being negatively biased or positively biased may be set using a chosen confidence level CL. In particular, sequences with a score >1−ε may be labelled as “positively biased”, whereas sequences with a score <c may be labelled as “negatively biased”, where ε is calculated as (1−CL)/2. For example, a confidence of CL=0.9975 represents a tolerance of 1 error in 400 tests (1/(1−0.9975), also referred to as 3Σ confidence). In embodiments, CL is at least 0.9975 (1 error in each 400 tests), at least 0.955 (1 error in each 22 tests, also referred to as 2Σ confidence) or at least 0.683 (1 error in each 3 tests, also referred to as 1Σ confidence). In embodiments, a fitness score is only calculated for a sequence variant if the sequence appears at least once in each of the first and second samples. This may be useful to exclude sequences that appear due to mistakes in the sequencing process and are not “true reads”. In embodiments, the scores are filtered to exclude sequence variants that appear less than a chosen number of times in the first sample, the second sample, or the sum of the first and second samples. For example, a threshold of minimum 4, 6, 8, 10, 15, or 20 reads in each sample or across both samples may be applied.

In embodiments, a separate bias score may be calculated for each sequence variant, for each desired functionality, as mentioned above. For example, assuming that the protein library is subjected to a first assay to quantify binding affinity to a first target, and a second assay to quantify binding affinity to a second target, two separate scores may be calculated, reflecting the bias of each of these assays in relation to each sequence variant.

At step 44, one or more machine algorithms are trained to build predictive models using the scores obtained in step 42. As such, models are obtained that relate features of the sequence of variants to fitness as measured by the scores obtained in step 42. In particular, where a plurality of fitness scores are calculated for each variant, a combined fitness score may be assigned for each variant and a single machine learning algorithm may be trained to build a predictive model based on the combined scores. Preferably, a plurality of machine algorithms may be trained, each based on one of the plurality of fitness scores. In other words, each algorithm may be trained to predict the fitness of sequences in relation to one desired functionality. In embodiments, a single (e.g. multivariate) model may be built to predict multiple fitness scores. In embodiments, the sequences of the variants may be encoded in a two or three dimensional matrix, and the fitness score for each variant (as a one dimensional vector) is used as a label. In embodiments, the variants are encoded at the amino acid or nucleotide level. Advantageously, encoding at the amino acid level may be significantly simpler than encoding at the base level, and may be appropriate to capture properties associated with the sequence of the protein (such as e.g. any property of the protein itself). In embodiments, the variants are encoded at the nucleotide level for some models (i.e. models trained to predict fitness scores associated with some desired functionalities), and at the amino acid level for other models (i.e. models trained to predict fitness scores associated with other desired functionalities). For example, sequences may be encoded in a two dimensional binary matrix also known as (hot-encoding) where each column corresponds to a position and variant at that position (e.g.: column 1: position 1-amino acid1; column 2: position 1-amino acid2, etc.) and each row corresponds to a variant (i.e. a variant that has amino acid2 at position 1 will have a 0 in column 1 and a 1 in column 2). In embodiments, sequences may be encoded in a three dimensional binary matrix (hot-encoding) where a first dimension (e.g. column) corresponds to a position, a second dimension (e.g. row) corresponds to a variant, and a third dimension (e.g. ‘depth’) corresponds to amino acids or nucleotides at the position, as the case may be. For example, a first column corresponds to position 1, a first row to variant 1, and the depth dimension to amino acids (depth1=amino acid 1, depth 2=amino acid 2, etc.). In this example, a variant that has amino acid 2 at position 1 will have a 0 at position (col1,row1,depth1), and a 1 at position (col11,row1,depth2) (and a 0 at every other position (col1,row1,depthx) where x is not 2). Alternatively, amino acids or nucleotides (as the case may be) may be numerically encoded and included in a matrix where each column corresponds to a position and each row to a variant. In such examples, a variant will have in each column of its row a number that represents the amino acid/nucleotide at the corresponding position.

In embodiments, one or more of the one or more machine learning algorithms is/are a classifier. In other words, the machine learning algorithm may be trained to predict which of a selected set of categories, a sequence is more likely to belong to. For example, categories of sequences may be defined as explained above as those that have a score labeled as “positive bias”, a score labeled as “negative bias”, and optionally a score labeled as “neutral”. The machine learning algorithm can then use the features of sequences assigned to each of these categories to learn what features are associated with the categories (either implicitly or explicitly), and predict the category of a new sequence. In embodiments where the machine learning algorithm is a classifier, the machine learning algorithm can be used to predict the class of any new sequence that it is provided, and/or to predict continuous values representing the probability for a new sequence that it is provided to belong to any of the defined classes. In embodiments where the machine learning algorithm is a regression algorithm, the machine learning algorithm can be used to predict a score for any new sequence that it is provided. In embodiments, the machine learning algorithm is a regression algorithm. In other words, the machine learning algorithm may be trained to predict a numerical value (e.g. a continuous numerical value) for each sequence. Classifiers may advantageously used when the data indicates that the bias scores strongly cluster around the ends of the range of scores (i.e. the majority of the sequence variants have a bias score close to 0 or close to 1). In embodiments where the machine learning algorithm is a classifier or regression algorithm, the algorithm may be a decision tree ensemble or support vector machine algorithm.

In embodiments, one or more machine learning algorithms may be used and the outputs of multiple algorithms may be compared or otherwise combined. In embodiments, the machine learning algorithm is a may be a deep learning algorithm. For example, the machine learning algorithm may be chosen from a dense neural network, a convolutional neural network, a recurrent neural network, an autoencoder, etc.

In embodiments, one or more of the one or more machine learning algorithms may be a so called “black box” algorithm such as a neural network classifier, for example a convolutional neural network or autoencoder. In embodiments, one or more of the one or more machine learning algorithms may advantageously be an interpretable model. Machine learning algorithms are used to capture differences between sequences that have the one or more desired properties, and those that do not. When the machine learning algorithm is a black box model (as are neural networks), it is typically not possible to extract the underlying sequence features that result in the classification directly from the model itself. However, the model is able to predict a score for any new sequence that is fed into the model. Further, interpretability techniques may be implemented to obtain additional insights into the data even when so called “black box” algorithms are used. For example, it may be possible to obtain some information about sequence features that are particularly important to the predictions made by a model by analysing the distribution of weights assigned to e.g. edges in a neural network, by testing feature importance and/or by implementing an attention mechanism to limit the number of factors taken into account at any one time. Advantageously, ‘white box’ or interpretable models may enable to directly extract patterns that underline the score behavior. Insights obtained either directly from the model or using interpretability techniques may be used to guide the step of designing new libraries, and/or to identify any features of the methods of the invention that would be advantageously adjusted. For example, insights from the machine learning models may help to identify flaws or biases in the design of an experimental step in the method. In embodiments, the one or more machine learning algorithms can be used to predict a class, score or probability of belonging to a class for an initial population of sequence variants. Preferably, the models built using the machine learning algorithms are able to provide a predicted score for a sequence variant, together with a measure of confidence in the prediction. In embodiments where multiple models are trained to predict multiple features of sequences, some of the knowledge embodied in the models can be shared between models. Without wishing to be bound by theory, it is believe that many features associated with protein function can be derived from high level features of structure of the protein. Therefore, such high level knowledge may advantageously be re-used between models. This may advantageously contribute to reduce the risk of overfitting the models to any particular feature and/or increase the efficiency of the model training process, In particular, in embodiments where neural network are used, some low level layers of a model may be re-used and the rest of the architecture may be built independently for each of the models predicting each individual feature. The model(s), or learnings derived therefrom, can be used to obtain a score a new population to be provided to the machine learning algorithms for scoring, with the ultimate objective of finding functionally improved sequence variants. In other words, the models, or learnings derived therefrom, trained on the data from the testing step 30 can be used to score variants, which may be used as a tool to search for improved variants as described below, at step 46.

At step 46, a search process is performed to identify new sequences or populations of sequences, the new sequences preferably having an improved fitness (per sequence or based on a summarised value at the population level), as predicted by the predictive models built at step 44, compared to the sequences or populations of sequences that have been tested thus far. The search process is typically iterative, whereby at each new iteration a new population is designed based on learnings from the previous iteration, the new population is assessed and new learnings are derived (e.g. the predictive models obtained at step 44 are improved) which are used in the next iteration (a process also referred to as “build-test-learn-design cycle”).

In embodiments, one or both of two types of search processes may be performed, which are referred to herein as a sequence search optimisation and a sequence-library search optimisation. Further, each of these types of search may be performed as an exhaustive search, or as a stochastic search. Exhaustive searches typically comprise generating and evaluating all possibilities in a search space. Stochastic searches typically rely on heuristic algorithms to explore the search space and identify optima in said space, as will be described further below. Exhaustive searches are typically only feasible for relatively small variant spaces, as enumeration and evaluation of all possible variants in large spaces is computationally expensive. Therefore, the choice between exhaustive and stochastic searches may depend on the size of the variant space to be searched, and the computational resources available.

In a sequence search optimisation, a population of sequences as a list of sequence variants is provided as an input to a search and optimisation algorithm (see below), and a new population of sequences as a list of sequence variants with improved fitness is provided as an output. In embodiments, the sequence search optimisation is exhaustive. In such embodiments, all of the possible sequence variants are individually evaluated using the predictive models generated at step 44 (i.e. a fitness score is predicted for each sequence and each property that is associated with a predictive model) and a subset of sequence variants with improved fitness may be selected. For example, a subset of sequence variants may be selected as the most highly ranked subset according to multi-objective criteria (as will be described further below). Alternatively, a sequence search optimisation may be stochastic, whereby a set of one or more sequence variants with improved fitness is obtained by iterative exploration of the search space from an initial set of one or more sequence variants. Genetic algorithms may be used for this purpose, as will be explained further below. In embodiments, one or more models are built at step 44 to predict each property of interest. For example, a plurality of models may exist which are able to predict the fitness scores of a tested library with similar levels of fit. Therefore, multiple models can be used to predict the fitness score of a sequence variant, and the outputs of these models can be aggregated to obtain a summarized value and a measure of uncertainty of this summarized value. For example, the average and standard deviation of the scores predicted for a sequence variant by a plurality of models (such as e.g. between 3 and 10, preferably between 5 and 10 models) trained at step 44 to predict the same property may be used as the score of the sequence variant.

In a sequence-library search optimisation, the optimisation process takes as input a frequency matrix including a column per amino acid or nucleotide (e.g. A, G, C, T) and a row per variable position, each cell comprising a frequency for the particular amino acid/nucleotide at the particular position. As such, the frequencies are typically between 0 and 1 and sum to 1 for each column. As the skilled person would understand, the frequency matrix constitutes an aggregate representation of a collection of sequences, the frequencies in the matrix being representative of the sequences in the collection. The use of a frequency matrix may be advantageous in the early stages of optimisation as they may enable to explore the sequence space more widely. When using an exhaustive search, multiple sequence libraries (frequency matrices) are generated, scored and compared to each other. Using a stochastic search, a list of one or more sequence library(ies) (frequency matrices) is provided as an input, each library is scored, and a new list of one or more improved libraries is selected. This can then be used as input for a new iteration of the search.

In order to score sequence-libraries (frequency matrices), the frequency matrix is used to generate a subset of sequences by sampling, the subset being considered to represent a “representative subset” of the library that is summarized in the frequency matrix. Each sequence in the subset is then scored as described above, using the models built in step 44. An aggregate value (also referred to as “summarized value”) may then be calculated as the score of the library, for each of the one or more fitness scores (i.e. for each of the one or more models trained). In embodiments, the aggregate value is the arithmetic average of the scores of the subset of sequences, or the n^{th} percentile of the scores of the subset of sequences (where n can be e.g. 50, 60, 70, 80 or 90), As explained above in relation to sequence search optimisation, this process may be repeated a number of times using multiple models trained at step 44 to predict the fitness of variants in relation to the same desired property(ies). An aggregated value across the subset-aggregated values predicted by each model, which includes a measure of variability of the subset aggregated values predicted, may therefore be calculated and used as a measure of fitness of the sequence-library.

The input (e.g. set of sequences or frequency matrix) to the optimisation process may be represented at the nucleotide level or at the amino acid level. Conducting the optimisation at the nucleotide level may be advantageous as a clearly defined many-to-one mapping exists between nucleotides and amino acids (via codons). By contrast, the reverse mapping may be less simple.

In embodiments, sequence search optimisation and sequence-library search optimisation may both be performed as part of a search process at step 46, for example at different iterations of the search process. In particular, sequence search optimisation and sequence-library search optimisation may be performed successively in order to balance exploration of the search space (wherein the search is adapted to encourage evaluation of new variants/regions of the search space) and exploitation of the learnings acquired through previous iterations of the search (wherein areas of the search space that are close to the currently known optimal region is searched in more detail). Typically, exploration is prioritized at the beginning of a search process (in which case this part of the process may be referred to as ‘exploration phase’), whereas exploitation is prioritized at the end of a search process (in which case this part of the process may be referred to as ‘exploitation phase’). In embodiments, sequence search optimisation is performed in the final iterations of a search process, in the exploitation phase. In embodiments, sequence-library search optimisation is performed at the beginning of a search process, in the exploration phase. Further, in the exploration phase, sequences or sequence libraries that are selected (as output of an exhaustive search or as input of a next iteration of a stochastic search) may be selected so as to prioritise sequences/sequence-libraries associated with high level of uncertainty in their predicted scores. Conversely, in the exploitation phase, sequence or sequence libraries may be prioritized based on lower levels of score uncertainty.

When all sequences (in a sequence search optimisation) or all sequence-libraries/frequency matrices (in a sequence-library search optimisation) have been scored, each sequence/sequence-library may be associated with a plurality of scores, where each score represents the predicted fitness of the sequence/sequence-library in relation to a desired property. Further, as explained above, each score may be associated with a measure of uncertainty, for example when the score is an aggregate of multiple scores predicted by multiple models built to predict fitness in relation to the same desired property. Therefore, the task of selecting a subset of top ranking sequences/sequence libraries (e.g. in the case of an exhaustive search or the last iteration of a stochastic search) or that of selecting a set of sequences/sequence-libraries for a subsequent iteration of a stochastic search algorithm is a multiobjective problem. In such embodiments, multiobjective optimisation algorithms may be used—where each objective may represent a fitness score representative of a desired property of a sequence variant or library. In embodiments, weights are applied to prioritise/emphasise some objectives (fitness scores) over others. In embodiments, multiobjective optimisation may be done using algorithms based on Pareto front optimisation, such as e.g. SPEA2 (Zitzler, Laumanns & Thiele, 2001, TIK-Report, volume 103, accessible using http:/www.research-collection.ethz.ch/handle/20.500.11850/145755 or https://doi.org/10.3929/ethz-a-004284029, which is incorporated herein by reference) or IBEA (Zitzler, Kunzli, 2004, Indicator-Based Selection in Multiobjective Search. In: Yao X. et al. (eds) Parallel Problem Solving from Nature—PPSN VIII. PPSN 2004. Lecture Notes in Computer Science, vol 3242. Springer, Berlin, Heidelberg, accessible using https://link.springer.com/chapter/10.1007/978-3-540-30217-9_84 or https://doi.org/10.1007/978-3-540-30217-9_84, which is incorporated herein by reference). Such algorithms may be able to reduce a full Pareto front population of solutions to a selected few solutions (sequences or sequence-libraries), while maximizing diversity (minimising overlap) between the selected solutions, for example by accounting for density considerations in the objective space. In embodiments, the optimisation may be designed to rank solutions highest if none of the objectives (fitness scores) can be improved in value without reducing the value of some of the other objectives (fitness scores). Such solutions represent a Pareto front. The optimisation process used may advantageously be designed to optimise the Pareto front, i.e. move the Pareto front towards higher values of the objectives (fitness scores) as the iterative optimisation progresses.

In embodiments, stochastic search methods are used to search a sequence variant space space. For example, stochastic searches may use a genetic algorithm. Briefly, the underlying principle is to calculate the fitness (i.e. score(s) or aggregated scores) of a population of individuals (where the individuals can be sequence variants in the case of sequence search optimisation, or sequence-libraries/frequency matrices in the case of sequence-library search optimisation), select a subset of the individuals of the population using at least in part the calculated fitness (and optionally using Pareto front algorithms as explained above), and subjecting the selected population to defined transformations to obtain a new population, which is then scored, etc. Applied to the present situation, an input set of sequences or frequency matrices is modified (i.e. subjected to transformations, such as mutation and/or cross-over with another individual, that are randomly selected according to predefined parameters), to obtain an initial population of sequences/matrices, referred to as the child population. This population is scored using the models trained in step 44. The child population is then pooled together with the input population, and a subset of this combined population is selected, for example by using a Pareto front optimisation algorithm as described above, which may in some embodiments rely on subjecting the population to a tournament style competition. Preferably, algorithms such as SPEA2 mentioned above are used which select the most diverse individuals in the Pareto front. The subset becomes the new initial population, and is modified as before to obtain a subsequent generation, which is similarly scored and selected. This process is repeated until a predefined stop criterion is met. For example, a stop criterion may be that a library with a sufficiently high fitness is generated, or a maximum, number of iterations is reached. Stopping parameters can be predefined by a user or can be assigned default values. In embodiments, transformations that can be applied to a population may be selected from mutations, crossovers, reproduction functions, etc.

In embodiments, the parameters of the genetic algorithm are optimised using methods known in the art. For example, genetic algorithm parameters such as population size, number of individuals in each children population crossover rate, mutation rate etc may be optimised using index based techniques such as BEA (Zitzler, Kunzli, 2004, https://link.springer.com/chapter/10.1007/978-3-540-30217-9_84, which is incorporated herein by reference). Such algorithms may advantageously enable to minimize fitness uncertainty in the exploitation phase and minimize it in the exploration phase, as explained above. Parameters of the genetic algorithm that are optimised may include one or more of: a choice of crossover strategy, crossover rate, mutation strategy, mutation rate, number of parents, population size, number of elites in the population, selection methods, etc. In embodiments, some parameters of the genetic algorithms may be adapted to take biological considerations into account, such as for example to address physical constraints or include domain knowledge in the search. For example, when the genetic algorithm operates at the nucleotide level, the mutation rates may be adapted to make a mutation in the first nucleotide of a codon less likely than a mutation on the second and/or third nucleotide of a codon. For example, a possible distribution of probability of mutation within a codon might be: 10%, 30%, 60%, for the first, second and third nucleotide, respectively, in each codon. In embodiments, the mutation and/or crossover parameters may be selected to exclude any sequence including a stop codon (e.g. TAG, TAA, TGA) in the translation phase of the sequence. In embodiments, the mutation and/or crossover parameters may be selected to exclude particular amino acids (either at the amino acid or at the corresponding codon level, depending on what level the optimisation algorithm operates at). Such exclusions may for example by defined by a user, based on prior knowledge. In embodiments, when performing cross-overs on sequence variants/sequence library variants, the cross-over point may be designed such that whole codons are exchanged between the variants.

In embodiments, the optimisation step may comprise running multiple optimisations in parallel and aggregating their outputs at intervals or at the ends of the runs. This may advantageously increase the diversity of the solutions obtained.

In embodiments, a distance is calculated between any new library that is generated and at least one previously generated library (e.g. any previously tested library and/or any previous in silico library). For example, a distance between a new library and a previously generated library may be used during a search process to prioritise exploration of the search space. Calculating the distance between previously generated libraries enables to assess the diversity of the libraries and ensure that the process is not limited to a specific area of the sequence space. In embodiments, the distance between sequence libraries is calculated using the Jensen-Shannon divergence method. The Jensen-Shannon Divergence (JSD) is a method of measuring the similarity between two probability distributions. In particular, the distributions can be discrete distributions. For example, the method can be used to calculate the distance between (1) a library where at a position p there is a 50% chance of having amino acid A1 and a 50% chance of having amino acid A2 (i.e. a probability vector of (A1, A2) equal to (50%, 50%)), and (2) a library where at position p there is a probability vector of (A1, A2, A3) equal to (40%, 40%, 20%). These two libraries have probability distributions P=(0.5, 0.5,0), and Q=(0.4, 0.4, 0.2)). The JSD is defined as JSD(P∥D)=λD(P∥M)+(1−λ)D(Q∥M) where M=λP+(1−λ)Q and λ is a weight selected between (0,1) (λ=0.5 for a symmetric case), and D(A∥B) is the Kullback-Leibler Divergence between the two distributions, i.e. DKL(A∥B)=−ΣiA(i) log(B(i)/A(i)). D(A∥B) (also called “relative entropy”) is a measure of how one probability distribution A differs from a base distribution B. For example, the base distribution B can be the initial library prior to optimisation using the machine learning algorithm, and the new library A can be the latest library generated by iterative optimisation. For each position p in each of the libraries, the value of JSD(Ap∥Bp) is calculated. The final divergence is then calculated as the sum of JSD over all positions p.

In embodiments, the distance between sequence libraries is calculated together with a significance term taking into account the likelihood of transitioning from one amino acid to another. In embodiments, the likelihood of transitioning from one amino acid to another is captured by a substitution matrix, such as a BLOSUM (Blocks Substitution Matrix), in particular BLOSUM62. BLOSUM is a matrix designed for alignment of protein sequences, and quantifies the probability of transitioning from one amino acid to another. For example, a significance associated with a divergence as calculated above can be calculated as described in Yona and Levitt (J Mol Biol. 2002 Feb. 1; 315(5):1257-75.) In particular, a significance is calculated as JSP(M∥BACKGROUND), where M is defined as before and BACKGROUND is a background signal. For example, a background signal may be chosen as the diagonal terms of BLOSUM62 (i.e. the likelihood of observing each amino acid). As such, a large significance means that P and Q are very different from the background signal, whereas a small similarity means that P and Q are similar to the background signal. Further, a similarity term may be calculated which takes into account both the divergence JSD(P∥Q) and the significance JSD(M∥BACKGROUND), and is defined as similarity=0.5*(1−D)*(1+S), where D is JSD(P∥Q) and S is JSD(M∥BACKGROUND). Therefore, the similarity is such that: (i) small D (D→0) and small S (S→0) values (P and Q are similar and not very different from the background) will result in similarities approaching 0.5 (Similarity→0.5); (ii) small D (D→0) and large S (S→1) values (P and Q are similar and very different from the background) will result in similarities approaching 1 (Similarity→1); and (iii) large D (D→1) values (P and Q are very different from each other) will result in similarities approaching 0 (Similarity→0).

In embodiments, a new library designed in step 16 may be built 20, tested 30 and used for a new learning phase 40. In such embodiments, the machine learning algorithm may be trained at step 42 using data from the current and previous iterations of the design-build-test process. In embodiments, a new library designed in step 16 may be used to produce a set of candidate proteins predicted to have the one or more desired properties.

In a specific embodiment of the invention, the described method can be implemented at least in part via one or more computer systems. In another embodiment the invention provides a computer readable medium containing program instructions for implementing at least the design 10,10′ and learn 40 phases of the method of the invention, and/or to control laboratory apparatus to implement the build 20 and test phases of the methods of the invention, wherein execution of the program instructions by one or more processors of a computer system causes the one or more processors to carry out the steps as described herein. Suitably, the computer system includes at least: an input device, an output device, a storage medium, and a microprocessor. Possible input devices include a keyboard, a computer mouse, a touch screen, and the like. Output devices computer monitor, a liquid-crystal display (LCD), light emitting diode (LED) computer monitor, virtual reality (VR) headset and the like. In addition, information can be output to a user, a user interface device, a computer-readable storage medium, or another local or networked computer. Storage media include various types of memory such as a hard disk, RAM, flash memory, and other magnetic, optical, physical, or electronic memory devices. The microprocessor is any typical computer microprocessor for performing calculations and directing other functions for performing input, output, calculation, and display of data. Two or more computer systems may be linked using wired or wireless means and may communicate with one another or with other computer systems directly and/or using a publicly-available networking system such as the Internet. Networking of computers permits various aspects of the invention to be carried out, stored in, and shared amongst one or more computer systems locally and at remote sites including within the cloud.

The methods of the invention may be configured to interact with and control automated laboratory equipment including liquid handling and dispensing apparatus or more advanced laboratory robotic systems. In embodiments, one or more steps are fully automated using a high-level programming language to produce reproducible and scalable workflows to underpin the design, testing and learning steps of the method. Suitable high-level programming languages may include C++, Python Java™, Visual Basic, Ruby and PHP, as well as the biology specific language Antha™ (www.antha-lang.org).

The invention is further illustrated by the following non-limiting examples.

### EXAMPLES

### Example 1—Engineering of a Scaffold Protein that Binds a Specific Target

In this Example, a library of sequence variants is generated based on a native sequence that has a binding affinity to a specific target. Based on the library, a collection of proteins that have improved binding affinity to the specific binding target compared to the native sequence is generated. This example demonstrates the use of the invention to produce a protein (or in this case a collection of candidate proteins) that has a desired functionality.

### Example 2—Selection of Protease Stable Variants

In this Example, a library of sequence variants (DNA) was designed semi-rationally based on structural information. The diversity of this initial library was around 3,000 variants. The library was assembled as described in WO 2017/046594 A1 (see below Materials and Methods). The library was inserted into a phage display vector, as known in the art, to be displayed on the outside of an M13 phage capsid after transformation in E. coli. The phage population, each displaying a protein variant of interest, was exposed to a protease (trypsin or chymotrypsin), resulting in cleavage of at least some protein variants. The pool of phages (both cleaved and uncleaved) was then exposed to an immobilized target protein, and any phages that failed to bind the target were washed away. The remaining phages (referred to a ‘round 1’ phages) were used to infect E. coli, producing a new phage population, some of which was used for selection as described above (resulting in a population of phages referred to as ‘round 2’ phages), and some of which was saved for sequencing. This process was repeated again to obtain a third population of phages, ‘round 3’ phages. Samples of DNA from each of the rounds and from the phage population prior to selection were prepared for next-generation sequencing using an NEBNext Ultra II DNA library prep kit for Illumina sequencing, as per the manufacturer's instructions. The samples were then sequenced using an Illumina iSeq sequencer. The sequence (Fastq files) from iSeq comprising forward and reverse reads were aligned to a reference sequence for the library using the Burrows-Wheeler Alignment algorithm. Paired-end reads were then merged using a consensus sequence to fill in any gap between paired ends, and resulting sequences were trimmed to remove ends that overhang the reference sequence and delete sequences that finish short of the reference sequence. The reads were then clustered using Starcode for error correction (as described in https://academic.oup.com/bioinformatics/article/31/12/1913/213875).

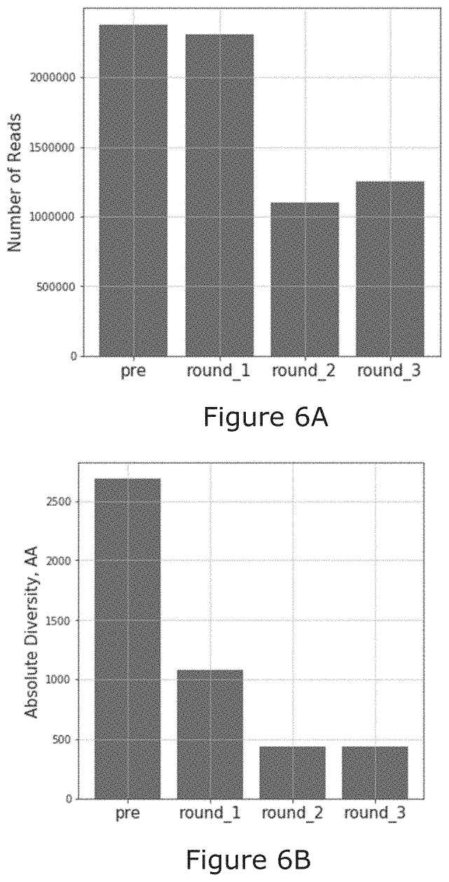

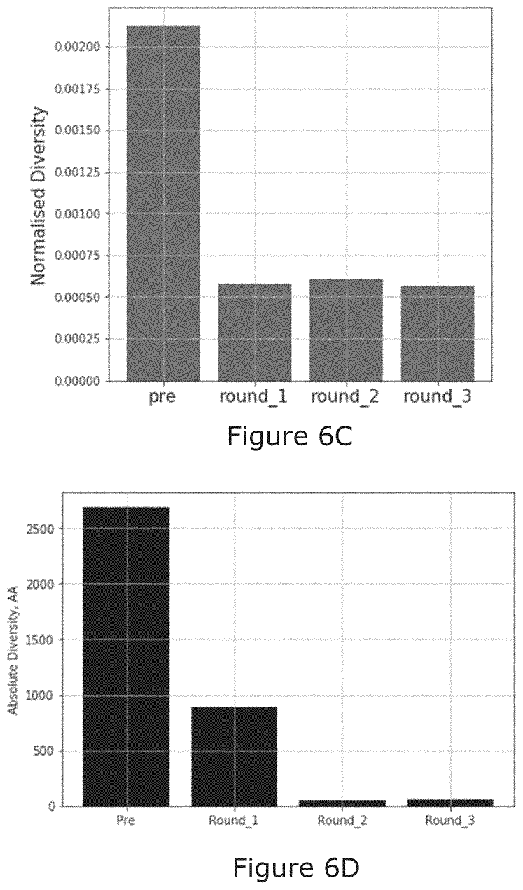

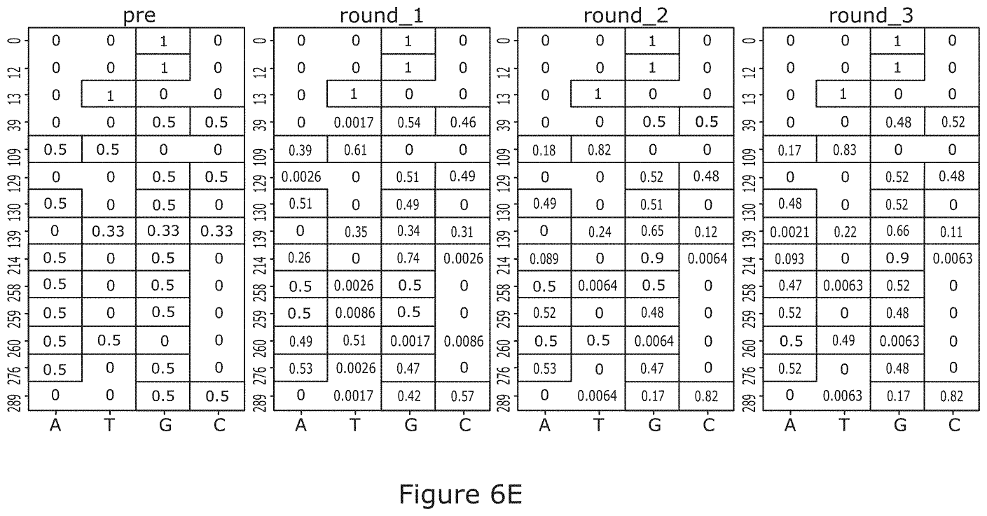

FIGS. 6A-6E show the results of this analysis. FIG. 6A shows the total number of raw reads in each sequencing run (prior to selection, labelled as ‘pre’ and after each round of selection, labelled as ‘round_1’, ‘round_2’ and ‘round_3’). FIG. 6B shows the total number of variants present in the population before selection (‘pre’) and after each round of selection. The data on FIG. 6B shows that the first round of selection dramatically reduces the number of variants sequenced (due to many variants being washed away during selection). The second round of selection further refines the population, while the third round does not appear to have a significant effect. The data on FIG. 6C shows the number of variants present in the population before selection (‘pre’) and after each round of selection, relative to the total number of reads (see FIG. 6A) for the corresponding sequencing run. The data shows that variants are represented by multiple reads even prior to selection, and that the number of reads per variant is further increased by selection (to a similar extent whether one, two or three rounds of selection were performed). FIG. 6D shows the total number of variants present in the population before selection (‘pre’) and after each round of selection, excluding any variants that were not present in the starting library. Comparing the data on FIGS. 6D and 6B shows that random mutations appear during the selection process, as the number of variants after selection (Figure E, ‘round_1’, ‘round_2’ and ‘round_3’), is higher than the number of variants in the corresponding data points on FIG. 6D, which are filtered to exclude variants not present in the original library.

FIG. 6E shows frequency tables showing the change in library composition at various variable positions before (‘pre’) and after each of 3 rounds of selection (‘round_1’, ‘round_2’ and ‘round_3’)—excluding those mutations that were not present in the original library.

This data demonstrates the feasibility of steps 12 to 32 of the present invention.

The inventors then repeated a similar experiment using mRNA display, in order to demonstrate the feasibility of such an option. Three DNA libraries encoding binding proteins were designed semi-rationally based on structural information. The diversity of these initial libraries was around 24,000 variants. The libraries were assembled as described in WO 2017/046594 A1 (see below Materials and Methods). These libraries were then displayed by mRNA display as described below (see Materials and Methods) to link their genotypes and phenotypes. This displayed library was then incubated with proteases—in this case, trypsin and chymotrypsin. After incubation with the proteases for 10 minutes and for 120 minutes, the reaction was halted and the proteins were purified via an N-terminal streptavidin binding tag. Once purified, the amount of full length protein was quantified by qPCR. Only full length, uncleaved proteins contain both the N-terminal strep tag, and the mRNA molecule at the C-terminus. Both the mRNA that was captured on the streptavidin beads, and the mRNA that was not captured were then amplified with qPCR. This allowed quantification of the amount of material present in both samples.

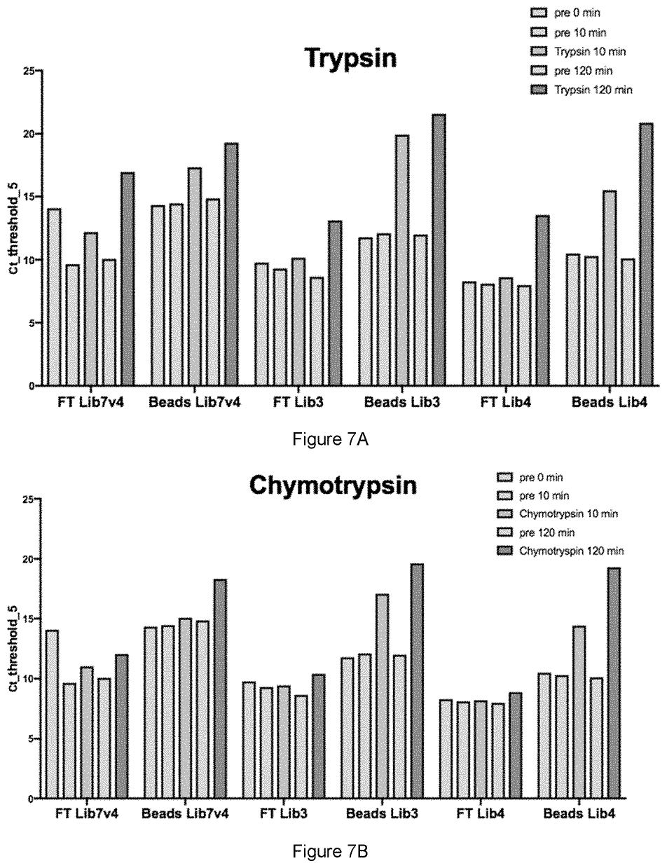

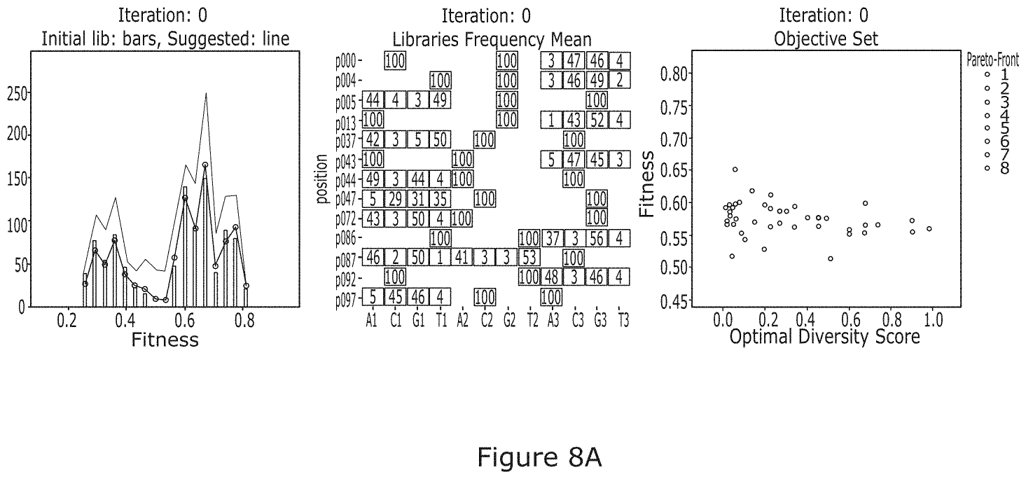

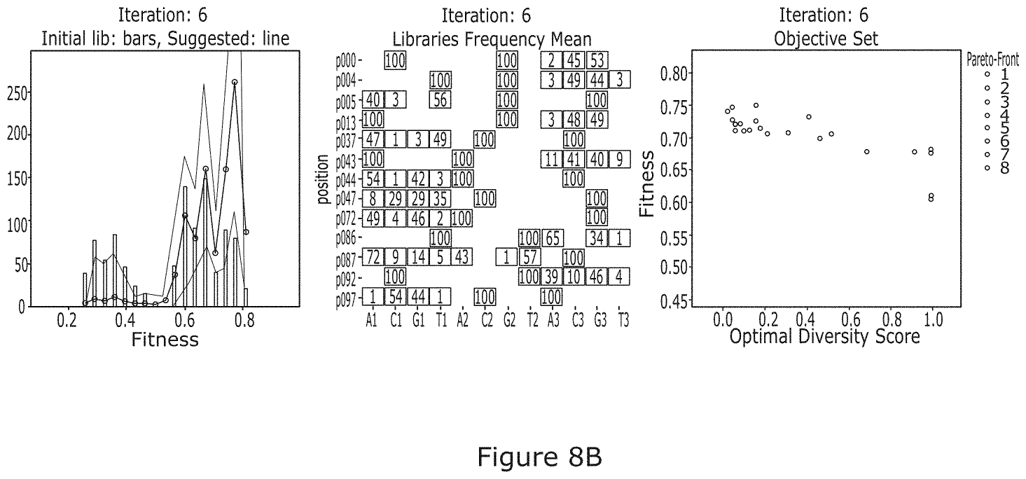

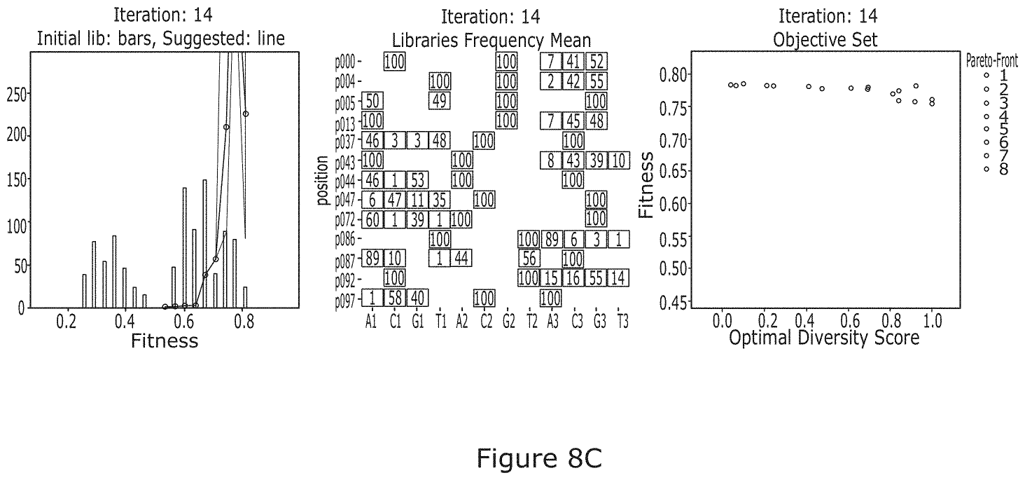

FIGS. 7A and 7B show the results of these analyses, for Trypsin (FIG. 8A) and Chymotrypsin (FIG. 8B), which show the results of the qPCR quantification (ct value, number of cycles at which the fluorescence signal reaches a level above background) for flow through samples (FT) and samples captured on the beads (Beads) for each of the three libraries. Each group of bars for each sample shows from left to right, the data for: sample prior to selection(pre), sample prior to selection after 10 minutes (pre 10 min), sample after 10 min selection ((Chymo)trypsin 10 min), sample prior to selection, after 120 minutes (pre 120 min), and sample after 120 min selection ((Chymo)trypsin 120 min). This data shows that the number of recovered sequences decreases when libraries are incubated with proteases, as expected. Further, the data shows that the decrease is dependent on incubation time (the decrease increases between 10 and 120 minutes of incubation with proteases). This demonstrates that using mRNA display and protease incubation, it is possible to enrich libraries for protease resistant molecules.

### Example 3—Sequence-Library Design by Iterative Optimisation

In this Example, a sequence-library is optimised in silico using a neural network classifier that has been trained on data obtained from in vitro testing of a library of sequence variants. In particular, publicly available immunogenicity data (from Dhanda et al., Front. Immunol. June 2018, available at https://www.frontiersin.org/articles/10.3389/fimmu.2018.01369/full) was used to train a predictive model for immunogenicity score, based on approximately 6,000 sequences. A set of sequence-libraries comprising 14 sequence-libraries was designed and scored using the neural network classifier trained on the in vitro data. Further, the diversity of each sequence-library was calculated and used as a second objective for optimisation. The diversity score was calculated as 1 for sequence-libraries with a diversity of 50,000 sequences, and lower than 1 for higher and lower diversity scores. In other words, one of the objectives of the optimisation algorithm was to design a library that is close to 50,000 variants, where the number of variants in a library is calculated by counting all possible combinations of variable positions. For example, a library that has two variable positions, each of which can be one of two amino acids has a diversity of 4 sequences, a library that has three variable positions each of which can be one of two amino acids has a diversity of 8 sequences, and so on. A subset of 10,000 sequences for each sequence-library was selected randomly with replacement, as a starting population for a genetic algorithm, which was run for a total of 80 iterations. The genetic algorithm was run until the maximum number of iterations (80) was reached, with 60 children at each generation, a crossover rate of 0.7 and a mutation rate of 0.3.

Each of FIGS. 8A to 8C illustrates an iteration of the optimisation process, as indicated. The left panel in each figure shows the distribution of fitness scores for the initial population (bars), and for the latest generation (dots and shaded area, where the dots are the mean values of the population score within each fitness histogram bin and the shaded area is the 2 standard deviations interval around the mean). The middle panel in each figure show the sequence-library in codon representation, where the rows are the position within the amino-acid sequence and the columns are the nucleotides within the codon (e.g, A1 is nucleotide A in the first base of the codon, where T3 is the T nucleotide in the third base in the codon). The values indicate the frequency (in %) each variant is represented at the nucleotide level. The right panel on each figure shows the pareto-front (maximum average fitness score for two separate parameters) for a number of libraries. As can be seen on these figures, the Genetic algorithm optimisation process enables to obtain a library that has an improved fitness score distribution by focusing on those variants that the machine learning algorithm (e.g. neural network) has identified as being associated with high fitness scores. As such, the members of this new library represent new sequence variants that are improved compared to the starting sequence in relation to the desired properties that were tested.

### Example 4—Using Machine Learning Driven Directed Evolution to Design Novel VHH Domains

In this example, a library of sequence variants (DNA) was designed semi-rationally based on mass spectrometry data of a VHH domain following incubation with a number of relevant protease enzymes. The diversity of this initial library was around 1×10⁹ variants. The library was assembled by Darwin assembly as described by Cozens et al, 2018 (Nucleic Acids Res. 46(8): e51). The library was inserted into a phage display vector, as known in the art, to be displayed on the outside of an M13 phage capsid after transformation in E. coli. The phage population was exposed to a target protein of interest, resulting in a number of protein variants binding to the target. Any phage particles that failed to bind the target were washed away. The remaining phage particles (referred to as ‘round 1’ phage) were used to infect E. coli, producing a new, enriched, phage population. This population was then used for selection as described above (resulting in a phage population referred to as ‘round 2’ phage). As well as the selected phage particle, mock control samples were generated that went through the same phage display steps, but were not selected against the target of interest. Samples of DNA from ‘round 2’ phage were prepared for next-generation sequencing via two PCR reactions—adding sequencing barcodes and adaptors, and purified using ProNex size-selective beads according to the manufacturers instructions. These samples were then sequenced using an Illumina MiSeq Sequencer.

DNA sequences (FastQ files) from the MiSeq Sequencer comprising forward and reverse reads were aligned to a reference sequence for the library using the Burrows-Wheeler Alignment algorithm. Paired-end reads were then merged using a consensus sequence to fill in any gap between paired ends, and resulting sequences were trimmed to remove ends that overhang the reference sequence and delete sequences that finish short of the reference sequence. The reads were then clustered prior to analysis and model training.

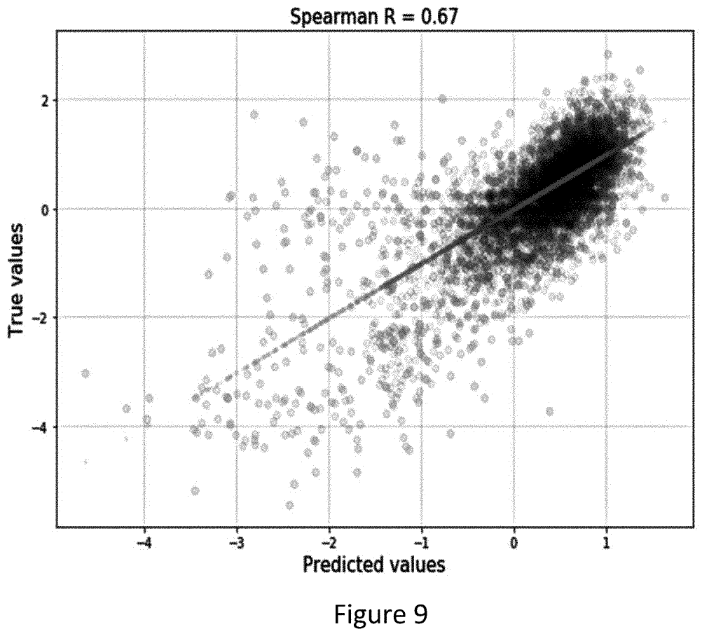

Each variant in the processed library was scored based on its enrichment during selection compared to the mock control. These scores along with the sequence information was used to produce a machine learning model that linked sequence to measured fitness. The accuracy of this model was assessed by comparing the predicted fitness of sequences that the model had not seen before, to their actual fitness. The correlation between the Spearman correlation between actual and predicted fitness for this model was 0.67, demonstrating the model is able to accurately predict binding to the target of interest based only on amino acid sequence (see FIG. 9).

### Example 5: In Vitro Validation of Binding Molecules

After using machine learning to predict a number of high performing variants, these variants were synthesised de novo using external gene synthesis suppliers. These genes were cloned into expression constructs and expressed with an E. coli chassis. Following expression, the candidate molecules were purified with an affinity tag. The affinity tags were then cleaved from the candidate molecules using a protease digestion.

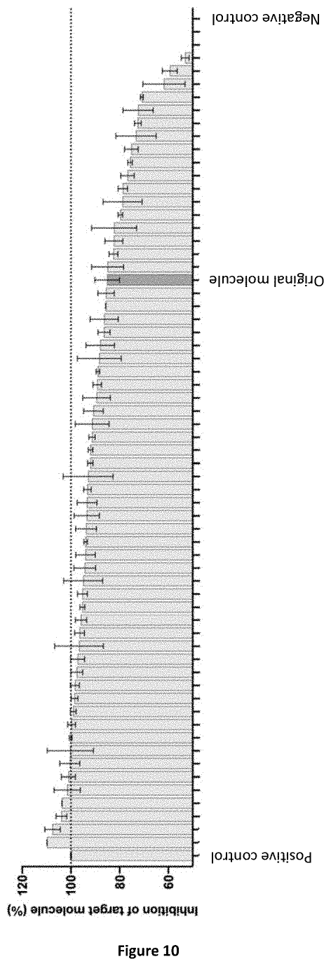

The performance of each molecule was measured using a cell-based potency assay. Following the assay, 68% of the molecules that the model predicted would have greater potency ended up doing so (see FIG. 10). This demonstrates that the accuracy of the model is retained in purified protein assays, as well as through NGS enrichment scores.

Materials and Methods

Single Primer Extension

Single primer extension may be used to obtain double stranded DNA from single stranded DNA molecules, for example variable parts of sequence variants in a library. In order to perform single primer extension according to embodiments of the invention, a single stranded DNA template is incubated with a short ssDNA sequence (called a primer) that is complimentary to the 3′ end of the template, and with a DNA polymerase. The sample is then subjected to the following incubation conditions:
98° C.—Melting: this step breaks any secondary structure that may have formed in the primer and ssDNA template;
55-70° C.—Primer annealing: allows the primer to anneal to (bind) the primer binding site at the 3′ end of the ssDNA template. The specific temperature may be dependent on the primer sequence.
72° C.—Extension: a DNA polymerase binds to the primer:template complex, and converts the rest of the ssDNA to dsDNA
4° C.—Store: keeps DNA from degrading once extension reaction is complete.

Compared to a polymerase chain reaction (see below), this differs in that: Template DNA is single stranded rather than double stranded; a single primer is used, rather than two; and the process is not cycled, so the template DNA is not amplified.

A single primer extension can be performed by hand or can be automated for example using Antha. In particular, the primer extension process used according to embodiments of the invention may be at least partially automated and divided in a plurality of steps including design, deck preparation, reaction setup, primer extension, purification and yield quantification.

In the primer extension design step, the identity of the primers used and the values of the parameters are defined. This can include an optimisation process wherein a search of at least a part of the parameter space is conducted to find the optimal parameter values for dsDNA yield.

In the deck preparation step, the deck of a liquid handling robot is prepared. This may include providing the individual component parts that are necessary for the reactions to be conducted, preparing a master mix of a subset of the components, and pipetting the mastermix and any other components into pre-defined locations of microtiter plates.

The core component parts of a primer extension reaction may include: one or more ssDNA templates, one or more ssDNA primers, a DNA polymerase, preferably a DNA polymerase with uracil read-through, such as Phusion U DNA polymerase, a polymerase buffer, dNTPs (deoxynucleotides triphosphate). In embodiments, other potential components can be added to primer extension reactions to optimise efficiency and fidelity. For example, any components selected from formamide, TMAC (trimellitic anhydride chloride), trehalose, CES (Combinatorial enhancer solution, see http://www.protocol-online.org/prot/Protocols/An-Economic-PCR-Enhancer-for-GC-Rich-PCR-Templates-3469.html), DMSO (dimethylsulfoxide), PEG (polyethylene glycol), ammonium sulfate, reverse transcriptases, mesophyllic DNA polymerases. DNA binding proteins, 7-deaza-2′-deoxyguanosine 5′-triphosphate, non-ionic detergents (Triton X-100, Tween 20, NP-40), and BSA (bovine serum albumin) may be added.

In the reaction setup step, all of the constituent components of the primer extension reaction are combined into mixtures ready for extension in the wells of one or more multiwell plate(s). In embodiments, this is carried out by a Gilson PIPETMAX liquid handling robot. This robot may be controlled by an Antha workflow.

In the primer extension step, the multiwell plate(s) is/are placed in a PCR machine or any other set up that is able to regulate the temperature of the plate. The samples in the plate are then subjected to the above-mentioned incubation conditions in order to carry out the extension reaction.

In the purification step, the molecules of dsDNA in each sample are separated out. In embodiments, this is performed by incubating the samples with magnetic beads that bind specifically to dsDNA, and “pulling down” the beads with a magnetic plate. The remaining reaction components can then be manually or automatically pipetted out.

In the yield quantification step, the amount of dsDNA produced is quantified using an assay as known in the art, for example a Picogreen assay and a Nanodrop or Tecan plate reader. Absorbance of light at 260 nm of the sample may be compared to standard curves to identify the amount of dsDNA in the sample.

Polymerase Chain Reaction

Polymerase Chain Reaction (PCR) may be used to amplify double stranded DNA, for example constant parts of sequence variants in a library. PCR may also be used to add deoxyuridine residues at specific locations in DNA parts. These may be used to generate single stranded overhangs by Uracil-Specific Excision (using a USER reagent).

In order to perform a PCR according to embodiments of the invention, a double stranded DNA template (which can form a part of a longer sequence) is incubated with two short ssDNA sequences (called primers) that are complimentary to the 3′ end of the respective strands of the template, and with a DNA polymerase. The sample is then subjected to the following incubation conditions:
98° C.—Melting: this step breaks the hydrogen bonds between the complementary strands of the DNA template, thereby allowing the primers to bind to their respective strands.
55-70° C.—Primer annealing: allows the primers to anneal to primer binding site at the 3′ ends of the template strands. The specific temperature may be dependent on the primer sequence.
72° C.—Extension: a DNA polymerase binds to the primer:template complex, and converts the rest of the ssDNA to dsDNA.

Repeat the above steps up to 35 times.
4° C.—Store: keeps DNA from degrading once extension reaction is complete.

A PCR can be performed by hand or can be automated for example using Antha. In embodiments, the PCR process used according to embodiments of the invention may be at least partially automated.

In embodiments, a PCR process may be divided in a plurality of steps including design, reaction preparation (optionally including deck preparation and reaction setup), thermocycling, purification and yield quantification.

In the PCR design step, the identity of the primers used and the values of the parameters are defined. This can include an optimisation process wherein a search of at least a part of the parameter space is conducted to find the optimal parameter values for target dsDNA yield.

One parameter that can be optimised is the primer annealing temperature. Different primer sequences may have different annealing temperatures. These annealing temperatures can be estimated with bioinformatics and/or can be elucidated by running a “gradient” annealing step. A gradient annealing step creates a range of temperatures across a thermocycler block, in order to test multiple different annealing temperatures in parallel to find out which temperatures provided the best target dsDNA yield.

In the reaction preparation step, all of the constituent components for a PCR are combined into mixtures ready for the reaction. This may be done by hand or using a liquid handling robot. In such embodiments, this may include a deck preparation step and a reaction setup step. In the deck preparation step, the deck of a liquid handling robot is prepared. This may include providing the individual component parts that are necessary for the reactions to be conducted, preparing a master mix of a subset of the components, and pipetting the mastermix and any other components into pre-defined locations of microtiter plates. In the reaction setup step, all of the constituent components of the PCR reaction are combined into mixtures ready for the PCR in the wells of one or more multiwell plate(s). In embodiments, this is carried out by a Gilson PIPETMAX liquid handling robot. This robot may be controlled by an Antha workflow.

The core component parts of a PCR may include: one or more dsDNA templates, one or more forward ssDNA primers, one of more reverse ssDNA primers, a thermostable DNA polymerase (e.g. preferably a DNA polymerase with uracil read-through, such as Phusion U DNA polymerase), a polymerase buffer, dNTPs (deoxynucleotides triphosphate). In embodiments, other potential components can be added to primer extension reactions to optimise efficiency and fidelity. For example, any components selected from formamide, TMAC (trimellitic anhydride chloride), trehalose, CES (Combinatorial enhancer solution, see http://www.protocol-online.org/prot/Protocols/An-Economic-PCR-Enhancer-for-GC-Rich-PCR-Templates-3469.html), DMSO (dimethylsulfoxide), PEG (polyethylene glycol), ammonium sulfate, reverse transcriptases, mesophyllic DNA polymerases. DNA binding proteins, 7-deaza-2′-deoxyguanosine 5′-triphosphate, non-ionic detergents (Triton X-100, Tween 20, NP-40), and BSA (bovine serum albumin) may be added,

In the thermocycling step, the multiwell plate containing the one or more samples is placed in a thermocycler or in any other setup capable of controlling the temperature of the sample(s) in the plate (e.g. any thermal cycling apparatus). The samples in the plate are then subjected to the above-mentioned incubation conditions in order to carry out the PCR.

An optional success verification test may be performed to ensure that the PCR was successful. This may include loading the samples onto an agarose gel, alongside a standard ladder comprising DNA fragments of known size, and performing an agarose gel electrophoresis, whereby DNA fragments migrate in the gel at a rate that is proportional to their size. The presence of a band on the gel at the expected size for the target DNA indicates that the PCR was successful.

In the purification step, magnetic beads may be used to separate out dsDNA, as explained above. This may be performed differently depending on whether a verification test was performed and whether the test indicates that a single dominant dsDNA product is present in the sample. If the verification test indicates that a single dominant dsDNA product is present in the sample, magnetic beads may be used to separate out dsDNA from the rest of the sample, as explained above. If more than one dsDNA products are present in the sample, a “Size Select” agarose gel may be used, wherein wells are pre-cut in the gel and filled with water, and the desired DNA migrates through the gel and into the well where it can be pipetted out.

In the yield quantification step, the amount of dsDNA produced is quantified using an assay as known in the art, for example a Picogreen assay and a Nanodrop or Tecan plate reader. Absorbance of light at 260 nm of the sample may be compared to standard curves to identify the amount of dsDNA in the sample.

Assembly

Assembly of nucleic acid libraries from variable and constant parts is performed as described in WO 2017/046594, the content of which is incorporated herein by reference.

In particular, USER DNA assembly may be used to assemble variable and constant parts that are to form sequence variants in a library.

In embodiments, a USER DNA assembly may be divided in a plurality of steps including design, reaction preparation (optionally including deck preparation and reaction setup), incubation, purification and yield quantification.

In the USER DNA assembly design step, the reaction mixtures and values of the parameters used are defined. This can include an optimisation process wherein a search of at least a part of the parameter space is conducted to find the optimal parameter values for target dsDNA yield.

In the reaction preparation step, all of the constituent components for a USER assembly are combined into mixtures ready for the reaction. This may be done by hand or using a liquid handling robot. In such embodiments, this may include a deck preparation step and a reaction setup step. In the deck preparation step, the deck of a liquid handling robot is prepared. This may include providing the individual component parts that are necessary for the reactions to be conducted, preparing a master mix of a subset of the components, and pipetting the mastermix and any other components into pre-defined locations of microtiter plates. In the reaction setup step, all of the constituent components of the reaction are combined into mixtures ready for incubation in the wells of one or more multiwell plate(s). In embodiments, this is carried out by a Gilson PIPETMAX liquid handling robot. This robot may be controlled by an Antha workflow.

The core component parts of a USER assembly may include: 2 or more input parts, a USER enzyme mix, a DNA ligase (e.g. T4 DNA ligase), a reaction buffer (e.g. a T4 DNA ligase buffer), and ATP.

In the incubation step, the microwell plate is placed in a thermoblock or nay other set up enabling the control of the temperature of the samples in the microwell plate (e.g. any thermal cycling apparatus). The incubation step may comprise a 37° C. step to allow the USER enzymes to perform their function, followed by a 21° C. step to allow the overhangs to anneal, and the DNA ligase to perform its function.

An optional success verification test may be performed to ensure that the assembly was successful. This may include loading the samples onto an agarose gel, alongside a standard ladder comprising DNA fragments of known size, and performing an agarose gel electrophoresis, whereby DNA fragments migrate in the gel at a rate that is proportional to their size. The presence of a band on the gel at the expected size for the target DNA indicates that the assembly was successful.

In the purification step, assembled dsDNA (i.e. dsDNA in the reaction product, that has the desired size), is separated from the rest of the reaction product. A “Size Select” agarose gel may be used for this, wherein wells are pre-cut in the gel and filled with water, and the desired DNA migrates through the gel and into the well where it can be pipetted out.

In the yield quantification step, the amount of dsDNA in the sample is quantified using an assay as known in the art, for example a Picogreen assay and a Nanodrop or Tecan plate reader. Absorbance of light at 260 nm of the sample may be compared to standard curves to identify the amount of dsDNA in the sample.

Darwin Assembly

Darwin assembly broadly consists of a 3-step process to introduce mutations into a template sequence. First, a double-stranded template DNA sequence is converted to a single stranded one. This is achieved by the coupled reaction of a nicking endonuclease and an exonuclease, followed by heat inactivation of enzymes.

This single stranded template is then mixed with a number of mutagenic oligonucleotides, as well as boundary oligonucleotides that flank the region of interest—one of which being labelled with a biotin tag. Once these oligonucleotides have annealed, the gaps between them are filled using a thermostable DNA polymerase and nicks are sealed with a thermostable DNA ligase. The assembled product is purified using streptavidin coated magnetic beads. This product is then amplified from the magnetic beads through the addition of “outnest” primers and a standard PCR reaction. This final product is ready to be cloned into a plasmid or used directly as a linear construct in in vitro display methods. #

Inverse PCR

Inverse PCR is performed using mutagenic oligonucleotides. These oligonucleotides anneal to the region of interest within a gene ‘back-to-back’, with one or both of the oligonucleotides containing a mutagenic region that is not complimentary to the template sequence. In the case of a substitution, this mutagenic region is positioned at the center or at the 5′ terminus of the mutagenic oligonucleotide. In the case of an addition mutation, the mutagenic region is positioned at the 5′ terminus of the oligonucleotide.

Once the mutagenic oligonucleotides have been mixed with the circular template dsDNA and the thermostable DNA polymerase, a conventional PCR reactions carried out. First, the sample is heated to >95° C. such that the dsDNA melts to ssDNA. The sample is then cooled to the annealing temperature of the primer (normally in the range of 55 to 65° C.) to allow the oligonucleotides to anneal to the template sequence. Once annealed, the sample is heated again, to the optimal extension temperature of the thermostable polymerase (for example, about 72° C.), and held there while the primers are extended. This process is cycled a number of times to produce sufficient yield (15 to 35× cycles).

Once the PCR reaction is complete, the DNA is purified using a PCR cleanup kit or a DNA agarose gel extraction. The template plasmid DNA is digested through the addition of a DpnI enzyme. The mutated PCR product is then recircularised with a DNA ligase, ready for transformation into a host cell.

Phage Display

First, the library of phagemid vectors are transformed into E. coli using electroporation. Following outgrowth on selective agar plates, the library of cells are scraped from the plates and resuspended in liquid media and glycerol, then stored.

These cells are then inoculated into a larger volume of liquid media and grown until mid-log phase. Once at mid-log, helper phage is added to the culture. The cells are grown for a further hour to allow infection with the helper phage.

Phage expression is then induced by pelleting the cells and re-suspending in induction media (containing IPTG). The cells are then grown overnight.

Phages are purified from the cells by centrifugation. The culture is spun at 5,000×g and the pellet discarded. The supernatant is then centrifuged at 11,000×g to pellet the phages. These pellets are the re-suspended in a storage buffer and can be stored at −80° C.

Once prepared, the phages can then be selected against a target. When selecting binders, the phages are exposed to a target molecule immobilized to a solid surface (such as a magnetic bead) at a specific concentration. Positive molecules bind to these target molecules, while the remaining variants do not. The surface is washed with a buffer to remove any variants that have bound non-specifically to the surface. After a number of wash cycles, the bound phages are eluted from the target.

Once eluted, some of the phage is separated and prepared for next generation sequencing. The rest is re-infected into E. coli, such that the positive variants are amplified and can be panned against the target again.

mRNA Display

mRNA display is performed as described in Barendt et al. (ACS Comb. Sci. 2013, 15, 2, 77-81; https://pubs.acs.org/doi/abs/10.1021/co300135r). Briefly, each member of the library is designed to contain a T7 promoter sequence upstream of the coding sequence. The DNA molecules are mixed with a T7 polymerase, buffer, and ribonucleotides triphosphate (rNTPs). The T7 polymerase binds the DNA template at the T7 promoter and transcribes the DNA to RNA. It continues to do this until it reaches a T7 terminator sequence at the 3′ end of the sequence, or it reaches the end of the linear DNA fragment. Once the reaction is complete, successful transcription is verified by gel analysis. The remaining reaction is treated with DNAse to remove the DNA template and then purified with Monarch® RNA cleanup columns (New England BioLabs, https://internation.neb.com/products/t2030-monarch-rna-cleanup-kit-10-ug#Product%20Information) to remove remaining salts, enzymes and rNTPs.

Each mRNA is then linked to a puromycin linker, which consists of a short DNA sequence, with a puromycin molecule at the 3′ terminus. A splint DNA sequence is used to efficiently ligate the puromycin linker to the 3′ terminus of each mRNA template. This splint sequence is complementary to both the 3′ end of the mRNA, and to the 5′ end of the puromycin linker. It therefore effectively brings the 3′ end of the mRNA and the 5′ end of the puromycin linker into close proximity. Once this has been achieved, a ligase (e.g. a T4 ligase) can be introduced to ligate these two molecules together. Once ligation is complete, the splint oligo is removed using DNA exonucleases, and the RNA is cleaned up, for example using Monarch® RNA clean up kits (New England BioLabs).

The mRNA-puromycin fusion molecules may then be translated, for example using the PURExpress® translation system (New England BioLabs; https://international.neb.com/products/e6850-purexpress-rf123-kit#Product%20Information). This cell-free mixture is a reconstituted protein expression system. All of the individual components that are required to express proteins are produced in cells, purified and mixed together. The main benefit of this system over other cell-free expression systems is that it is very clean; containing few RNAses.

Once the translation is complete, the reaction conditions are altered to encourage the puromycin fusion to occur—this involves cooling the samples and increasing the salt concentration.

The fusion molecules are then quality controlled either by Northern Blot or by quantitative PCR (qPCR).

For the Northern Blot, samples are run on an RNA gel (e.g. tris-borate urea gel), and blotted onto a nylon membrane. Digoxigen (DIG)-modified RNA oligos are then hybridised to the RNA on this membrane. Once this is complete, the DIG-labelled mRNA can be detected using the protocol defined in a DIG luminescent detection kit:

(Sigma Aldrich, https://www.sigmaaldrich.com/catalog/product/ROCHE/11363514910?lang=en&region=GB).

This process separates and visualises the mRNA in the sample. In a successful mRNA display, 3 bands should appear: one for just the mRNA, another for the mRNA-puromycin and a third for the mRNA-puromycin-protein fusion (this being the largest of the three).

For the qPCR, the variants in the library are designed to contain a strep-tag sequence or streptavidin binding peptide sequence (or other purification tag) such that the proteins include the purification tag. The expressed proteins are then separated out using the appropriate affinity separation method, for example streptavidin labelled magnetic beads, optionally by incubating the samples with a blocking agent such as heparin. Quantitative reverse transcription PCR, as known in the art, is then performed to quantify the amount of mRNA present in the sample. With successful mRNA display, the amount of RNA present in the sample should be much higher compared to a negative control. As a negative control, a protein sample (e.g. a matching protein library) that does not contain the puromycin linking the mRNA to the protein may be used.

Reverse Transcription

Prior to sequencing of the sequence variants that have been separated into groups depending on their behaviour in one or more functional assays, the mRNA sequences attached to the protein variants may be reverse transcribed to obtain a DNA sample that is representative of the variants in each group that is to be sequenced. This is performed as known in the art, by incubating the samples with a reverse transcriptase, a primer, a suitable buffer and dNTPs.

Next Generation Sequencing

Next Generation Sequencing (NGS) according to embodiments of the invention is performed using Illumina sequencers. As such, a sample to be sequenced may be prepared for sequencing by including DNA adaptors. The DNA adaptors may include a region that is used to bind the DNA sequences to the sequencing chip, a region that allows a primer sequence to bind to the sequence, and optionally a barcode sequence which allows different groups of variants to be sequenced together.

Illumina sequencing and library preparation for Illumina sequencing are known in the art. For example, library preparation for sequencing can be performed using the NEBNext Kits (New England Biolabs), as described in https://www.neb.com/-/media/nebus/files/brochures/nebnextillumina.pdf (pages 4 and 5).

Embodiments of the invention use an Illumine iSeq 100 sequencer. This sequencer is currently able to produce around 5 million 2×150 reads in 17 hours.

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

What is claimed is:

1. A method for producing a protein having one or more desired properties, the method comprising:
(a) a library design step, in which a nucleic acid library comprising at least 10⁴ sequence variants is designed, wherein each sequence variant comprises a coding sequence fora protein and each sequence variant comprises at least one constant region and at least one variable region, wherein one or more constant regions are common to all sequence variants within the library, and the one or more variable regions are not common to all sequence variants within the library;
(b) a library testing step, in which the sequence variants are tested in parallel, for the one or more desired properties; and
(c) a learning step, in which the sequence variants are each assigned a fitness score based at least in part on the result of the library testing step, and a machine learning algorithm uses the fitness score of each of the sequence variants to train a model to predict the fitness score for new sequence variants;
wherein the machine learning model trained in step (c) is used to design a new library of sequence variants with an improved distribution of fitness scores.

2. The method of claim 1, further comprising: (a′) a library assembly step, comprising:
providing a first plurality of nucleic acid molecules corresponding to a first variable part of the sequence variants in the library, comprising one or more variable regions, and wherein the first plurality of nucleic acid molecules comprises variants of the one or more variable regions;
providing:
at least one further pluralities of nucleic acid molecules corresponding to at least one further variable part of the sequence variants in the library, comprising at least one further variable region wherein the at least one further plurality of nucleic acid molecules comprises variants of the at least one further variable regions; and/or
at least one further plurality of nucleic acid molecules corresponding to a at least one constant part of the sequence variants in the library, each constant part comprising a constant region and no variable region, wherein the at least one further plurality of nucleic acid molecules are substantially identical;
assembling each of the plurality of first and at least one further nucleic acid molecules to form the nucleic acid library, each variant in the library comprising a first variable part and at least one further part.

3. The method of claim 1 or 2, wherein the library design step (a) utilises USER assembly, Darwin assembly and/or inverse PCR.

4. The method of claim 2, wherein the nucleic acid molecules corresponding to each of the one or more variable parts are provided as single stranded DNA, optionally wherein providing a plurality of nucleic acid molecules corresponding to the variants of one or more variable parts comprises synthesising a second DNA strand by single primer extension to form double stranded DNA.

5. The method of any preceding claim, wherein constant parts are up to about 2000 nucleotide long, and/or wherein variable parts are up to about 200 nucleotide long.

6. The method of any preceding claim, wherein each sequence variant comprises a plurality of constant parts and/or a plurality of variable parts.

7. The method of any preceding claim, wherein the library design step (a) comprises designing at least one of the one or more variable regions to include random variability in at least one position, optionally wherein the library design step (a) comprises designing at least one of the one or more variable regions to include random variability in one or more specific positions of the at least one variable region.

8. The method of claim 7, wherein including random variability comprises constraining the variability to sequences that correspond to a DNA codon.

9. The method of any preceding claim, wherein the library design step (a) comprises:
selecting a nucleic acid sequence encoding for a protein that has at least one of the one or more desired properties;
automatically identifying one or more regions of the sequence where variability is expected to result in an improvement of the at least one of the one or more desired properties and/or acquisition of at least one of the one or more desired properties; and
defining the one or more variable parts to include the one or more regions of the sequence where variability is expected to result in an improvement of the at least one of the one or more desired properties and/or acquisition of at least one of the one or more desired properties.

10. The method of claim 9, wherein the library design step (a) further comprises: identifying one or more regions of the sequence where variability is expected to be detrimental to the integrity of the protein and/or to at least one of the one or more desired properties; and defining one or more of the one or more constant regions to include the one or more regions of the sequence where variability is expected to be detrimental to the integrity of the protein and/or to at least one of the one or more desired properties.

11. The method of any preceding claim, wherein at least one of the one or more constant regions comprises one or more sequences selected from: a promoter sequence, an enhancer sequence, a localisation signal, a flag sequence, a marker sequence, a ribosome binding site, a stop codon, a start codon, a 5′ stem loop structure, a 3′ stem loop culture, an origin of replication and a selection sequence.

12. The method of any preceding claim, further comprising a step (a″) of producing the proteins encoded by each sequence variant of the nucleic acid library to obtain a protein library, wherein the library testing step (b) comprises subjecting the protein library to one or more assays to test for the one or more desired properties.

13. The method of claim 12, wherein the nucleic acid library is a DNA library and producing the protein library comprises transcribing and translating the DNA library, wherein translating the library comprises synthesising RNA-polypeptide fusion molecules each comprising an RNA sequence variant bound to the protein that it encodes.

14. The method of claim 12, wherein the nucleic acid library is a DNA library and producing the protein library comprises transcribing and translating the DNA library, wherein translating the library comprises propagating phage that display a coat protein-polypeptide fusion, wherein the polypeptide fused to the coat protein corresponds to a sequence variant of the DNA library.

15. The method of claim 12 or claim 13 or claim 14, wherein the library testing step (b) comprises separating the protein library into at least 2 samples depending on the results of the one or more assays, and sequencing the nucleic acids present in at least one of the at least 2 samples.

16. The method of claim 15, wherein the learning step (c) comprises aligning the sequences obtained by sequencing with the sequences designed in step (a), and quantifying the number of times that each sequence appears in each sample.

17. The method of any preceding claim, wherein the one or more desired properties is/are chosen from: physico-chemical properties of the proteins, activity-related properties, physiologically-relevant properties, and pharmacokinetic properties.

18. The method of claim 17, wherein at least one of the constant regions comprises a sequence that encodes for a protein purification tag, optionally wherein the protein purification tag is located at the C terminus of the protein, wherein one of the one or more desired properties is protease resistance and running the protein library through one or more assays comprises exposing the protein library to one or more proteases, purifying the proteins using the protein purification tag and identifying the sequence variants that are not cleaved by the one or more proteases.

19. The method of claim 15 or 16 to 18 when dependent on claim 15, wherein one of the one or more desired properties is binding to a specific target, and the library testing step (b) comprises incubating the protein library with the specific target immobilised on a surface and separating the protein library into a sample that is bound to the surface and a sample that is not bound to the surface.

20. The method of any preceding claim, wherein the library testing step comprises testing the variants for a plurality of properties, and the learning step comprises assigning a plurality of fitness scores to each variant tested, wherein each fitness scores corresponds to one of the plurality of properties, wherein the learning step comprises training a plurality of machine learning algorithms, wherein each machine learning algorithm is trained to predict at least one of the plurality of fitness scores for new sequence variants.

21. The method of claim 16 or any of claims 17 to 20 when dependent on claim 16, wherein the one or more fitness scores associated with each sequence variant depends on the number of times that each sequence appears in a first sample and the number of times that each sequence appears in a second sample, optionally wherein the first sample corresponds to a sample that is deemed to have a positive result in one of the one or more assays, and the second sample is a control sample.

22. The method of any preceding claim, wherein the machine learning algorithm is a classifier, wherein the machine learning algorithm is a neural network.

23. The method of any preceding claim, wherein the machine learning model trained in step (c) is used to design a new library of sequence variants by iteratively optimising a library of sequence variants in silico, optionally wherein the library of sequence variants is iteratively optimised using a genetic algorithm.

24. The method of any preceding claim, further comprising repeating steps (a) to (c) with the new library.

25. The method of any preceding claim, wherein the new library comprises at least one sequence variant encoding for a protein with the one or more desired properties.

26. The method of any preceding claim, wherein the new library of sequence variants with an improved distribution of fitness scores is one wherein at least 30% of the sequence variants have one or more variable regions having a DNA sequence similarity of less than 95% with respect to the corresponding one or more variable regions of all, or a proportion of, the sequence variants within the library prepared in step (a).

27. The method of any preceding claim, wherein a higher proportion of sequence variants of the new library display one or more improved desirable properties compared to the sequence variants within the library prepared in step (a).

28. A system for producing a protein having one or more desired properties, the system comprising:
(i) a processor adapted to implement the method of any of claims 1 to 27;
(ii) a laboratory automation apparatus, wherein the apparatus is controlled by the processor so as to implement at least the testing step.

29. The system of any of claim 28, wherein the laboratory automation apparatus comprises one or more of the group consisting of: liquid handling and dispensing apparatus; container handling apparatus; a laboratory robot; an incubator; plate handling apparatus; a spectrophotometer; chromatography apparatus; a mass spectrometer; thermal-cycling apparatus; nucleic acid sequencing apparatus; and centrifuge apparatus.
